(19) United States
(12) Patent Application Publication
(10) Pub. No.: US 2009/0010865 A1
(43) Pub. Date: Jan. 8, 2009

(54) Process for Preparing Polymers in Aqueous Solvents

(75) Inventors: Son Nguyen Kim, Hemsbach (DE); Marianna Pierobon, Ludwigshafen (DE); Gabi Winter, Shanghai (CN); Matthias Laubender, Schifferstadt (DE)

Correspondence Address: CONNOLLY BOVE LODGE & HUTZ LLP, 1875 EYE STREET, N.W., SUITE 1100, WASHINGTON, DC, 20006, US

(73) Assignee: BASF SE, Ludwigshafen (DE)

(21) Appl. No.: 12/161,889

(22) PCT Filed: Jan. 15, 2007
(86) PCT No.: PCT/EP07/50357

§ 371 Date: Jul. 23, 2008

(30) Foreign Application Priority Data
Jan. 23, 2006 (DE) 06100731.6

(51) Int. Cl. A61K 31/74

(52) U.S. Cl. 424/70.11

(57) ABSTRACT

The present invention relates to methods of producing polymers which comprise, in copolymerized form, 50-95% by weight of at least one ester of (meth)acrylic acid, 5-50% by weight of at least one olefinically unsaturated, free-radically polymerizable anionogenic or anionic compound and 0 to 30% by weight of at least one further free-radically polymerizable compound by free-radical polymerization in an alcohol-comprising solution, where the polymerization initiator used is at least one water-soluble initiator.

The present invention relates to methods of producing polymers which comprise, in copolymerized form, 50-95% by weight of at least one ester of (meth)acrylic acid, 5-50% by weight of at least one olefinically unsaturated, free-radically polymerizable anionogenic or anionic compound and 0 to 30% by weight of at least one further free-radically polymerizable compound by free-radical polymerization in an alcohol-comprising solution, where the polymerization initiator used is at least one water-soluble initiator.

Numerous methods of producing polymers for hair cosmetics are known from the prior art. These include, for example, solution polymerization, precipitation polymerization, suspension polymerization or emulsion polymerization.

EP-A 0 694 565 describes a method for the homogeneous polymerization of water-insoluble polymers which comprise more than 50% by weight of monomers chosen from the group consisting of C1-C18-alkyl acrylate or -methacrylate esters, N-substituted acrylamides or methacrylamides and mixtures thereof, in essentially nonaqueous organic solvents, wherein the polymerization initiator used is a water-soluble initiator which is dissolved in an amount of water sufficient for dissolving the initiator and where the polymer obtained has lower residual monomer contents than are obtained using equivalent amounts of water-insoluble initiators. The solvent used comprises at most as much water as is necessary to keep the water-soluble initiator in dissolved form.

WO 94/24986 describes the preparation of hair-setting polymers based on acrylic acid and acrylic esters by solution polymerization in ethanol. Polymerization in alcoholic solvent mixtures which comprise water in the range from more than 25 to 50% by weight is not described.

EP-A 0 379 082 describes the preparation of hair-setting polymers based on acrylic acid and acrylic esters by solution polymerization in ethanol. Polymerization in alcoholic solvent mixtures which comprise water in the range from more than 25 to 50% by weight is not described.

The methods known from the prior art often have the disadvantage that the reaction times are long, particularly in the case of polymers comprising acrylic acid, and that the resulting polymers sometimes have residual monomer contents.

For ecological considerations, it is desirable to keep the use of volatile organic components (volatile organic compounds, VOC) as low as possible (low VOC). With regard to the economic feasibility of methods, it is also desirable to use readily available and favorable feed materials.

The object of the present invention was to provide an improved method of producing polymers suitable for cosmetic applications which overcomes the abovementioned disadvantages of the known methods.

This object was achieved through a method of producing polymers which comprise, in copolymerized form,
i) 50-95% by weight of at least one-ester of (meth)acrylic acid,
ii) 5-50% by weight of at least one olefinically unsaturated, free-radically polymerizable anionogenic or anionic compound and
iii) 0 to 30% by weight of at least one further free-radically polymerizable compound,
   by free-radical polymerization in an alcohol-comprising solution, where the polymerization initiator used is at least one water-soluble initiator, wherein the polymerization solution comprises water in the range from more than 25 to 50% by weight.

The polymers produced by the method according to the invention are preferably sparingly soluble or insoluble in water. At 20° C. and 1013 mbar, the polymers are preferably soluble to at most 10 g in 1 liter of water.

### i) Esters of (Meth)Acrylic Acid

Suitable esters of (meth)acrylic acid are, for example methyl (meth)acrylate, ethyl (methyacrylate, n-propyl (meth)acrylate; isopropyl (meth)acrylate, n-butyl (meth)acrylate, isobutyl (meth)acrylate, sec-butyl (meth)acrylate, 2-pentyl (meth)acrylate, 3-pentyl (meth)acrylate, isopentyl (meth)acrylate, neopentyl (meth)acrylate, n-octyl (meth)acrylate, 1,1,3,3-tetramethylbutyl (meth)acrylate, ethylhexyl (meth)acrylate, n-nonyl (meth)acrylate; naecyl (meth)acrylate, n-undecyl (meth)acrylate, tridecyl (meth)acrylate, myristyl (meth)acrylate, pentadecyl (meth)acrylate, palmityl (meth)acrylate, heptadecyl (meth)acrylate, honadecyl (meth)acrylate, arachinyl (meth)acrylate, behenyl (meth)acrylate, lignocerenyl (meth)acrylate, cerotinyl (meth)acrylate, melissinyl (meth)acrylate, palmitoleinyl (meth)acrylate, oleyl (meth)acrylate, linoleyl (meth)acrylate, linolenyl (meth)acrylate, stearyl (meth)acrylate, lauryl (meth)acrylate, phenoxyethyl (meth)acrylate, 4-t-butylcyclohexyl acrylate, cyclohexyl (meth)acrylate, ureido (meth)acrylate, tetrahydrofurfuryl (meth)acrylate and mixtures thereof.

The polymers obtainable by the method according to the invention comprise 50 to 95% by weight, preferably 65-85% by weight and in particular 70-80% by weight of component i) in copolymerized form.

### ii) Anionogenic or Anionic Compounds

Compound II) is an olefinically unsaturated, free-radically polymerizable anionogenic or anionic compound. Here, an anionogenic compound is understood as meaning a compound which can be converted into the corresponding anionic form by deprotonation with customary, preferably cosmetically acceptable organic or inorganic bases.

Compound II) is preferably chosen from the group of olefinically unsaturated, free-radically polymerizable carboxylic acids and salts thereof.

Compound II) is particularly preferably chosen from the group consisting of acrylic acid, methacrylic acid, ethacrylic acid, alpha-chloroacrylic acid, crotonic acid, maleic acid, maleic anhydride, itaconic acid, citraconic acid, mesaconic acid, glutaconic acid, aconitic acid, fumaric acid, half-esters of olefinically unsaturated dicarboxylic acids having 4 to 10, preferably 4 to 6, carbon atoms and salts thereof.

Compound II) is very particularly preferably chosen from the group consisting of acrylic acid, methacrylic acid, salts thereof and mixtures thereof.

The polymers obtainable by the method according to the invention comprise 5-50% by weight, preferably 10-40% by weight and particularly preferably 15-28% by weight of component ii) in copolymerized form.

iii) Further Free-Radically Polymerizable Compound

Suitable as compound iii) are generally all free-radically polymerizable compounds different from i) and ii). Preferred compounds iii) are chosen from the following groups:

iii1) free-radically polymerizable compounds comprising urethane groups
iii2) (meth)acrylamides,
iii3) cationogenic monomers,
iii4) cationic monomers,
iii5) compounds with at least 2 polymerizable double bonds, which are also usually referred to as crosslinkers,
iii6) compounds containing amide groups that are different from iii2) to iii4)
iii7) polyesters comprising at least two free-radically polymerizable, olefinically unsaturated double bonds
iii8) polyethers comprising at least two free-radically polymerizable, olefinically unsaturated double bonds
iii1) Free-Radically Polymerizable Compounds Comprising Urethane Groups

At least one olefinically unsaturated compound containing urethane groups can be used as component iii1). For the purposes of the present invention, olefinically unsaturated compounds containing urethane groups are understood as meaning compounds which comprise at least one urethane group and at least one polymerizable, preferably free-radically polymerizable, olefinic double bond. Olefinically unsaturated prepolymers comprising urethane groups suitable as component iii1) for the polymers obtainable by the method according to the invention are given, for example, in P. K. T. Oldring (Ed.), Chemistry and Technology of UV- and EB-Formulations for Coatings, Inks and Paints, Vol. 11, SITA Technology, London, 1991, pp. 73-123, to the entire contents of which reference is hereby made. (Poly)urethane (meth)acrylates are known to the person skilled in the art. They can be obtained by reacting a di- or polyisocyanate with a chain extension agent from the group of diols/polyols and/or diamines/polyamines and/or dithiols/polythiols and/or alkanolamines and subsequently reacting the remaining free isocyanate groups with at least one hydroxyalkyl (meth)acrylate or hydroxyalkyl ester of other ethylenically unsaturated carboxylic acids. The amounts of chain extension agent, di- or polyisocyanate and hydroxyalkyl ester are preferably chosen here so that

1.) the equivalent ratio of the NCO groups to the reactive groups of the chain extension agent (hydroxyl, amino or mercaptyl groups) is between 3:1 and 1:2, preferably 2:1, and
2.) the OH groups of the hydroxyalkyl esters of the ethylenically unsaturated carboxylic acids are present in stoichiometric amounts with regard to the still free isocyanate groups of the prepolymer of isocyanate and chain extension agent.

Furthermore, it is possible to produce (poly)urethane (meth)acrylates by firstly reacting some of the isocyanate groups of a di- or polyisocyanate with at least one hydroxyalkyl ester, and then reacting the remaining isocyanate groups with a chain extension agent. In this case too, the amounts of chain extension agent, isocyanate and hydroxyalkyl ester are chosen so that the equivalent ratio of the NCO groups to the reactive groups of the chain extension agent is between 3:1 and 1:2, preferably 2:1 and the equivalent ratio of the remaining NCO groups to the OH groups of the hydroxyalkyl ester is 1:1. All intermediate forms of these two methods are of course also possible. For example, some of the isocyanate groups of a diisocyanate can firstly be reacted with a diol, then more of the isocyanate groups can be reacted with the hydroxyalkyl ester and afterwards the remaining isocyanate groups can be reacted with a diamine. These different preparation methods of the polyurethane (meth)acrylates are known (e.g. from EP-A 0 203 161) and therefore require no detailed description.

Urethane (meth)acrylates suitable as component iii1) are also described in DE-A 198 38 852 p. 3, I. 45 to p. 9, I. 20, to the entire contents of which reference is made at this point.

Urethane (meth)acrylates are understood as meaning compounds which comprise, in copolymerized form,
A) at least one compound which comprises at least one active hydrogen atom and at least one free-radically polymerizable, α,β-ethylenically unsaturated double bond per molecule,
B) at least one diisocyanate and
C) at least one compound which comprises two active hydrogen atoms per molecule,
   and the salts thereof.

### Component A)

Suitable compounds A) are, for example, the customary vinyl compounds known to the person skilled in the art which additionally have at least one group that is reactive toward isocyanate groups and is preferably chosen from hydroxyl groups and primary and secondary amino groups. These include, for example, the esters of α,β-ethylenically unsaturated mono- and dicarboxylic acids with at least dihydric alcohols. α,β-Ethylenically unsaturated mono- and/or dicarboxylic acids which can be used are, for example, acrylic acid, methacrylic acid, fumaric acid, maleic acid, crotonic acid, itaconic acid etc. and mixtures thereof. Suitable alcohols are customary diols, triols and polyols, e.g. 1,2-ethanediol, 1,3-propartediol, 1,4-butanediol, 1,5-pentanediol, 1,6-hexanediol, 1,10-decanediol, diethylene glycol, 2,2,4-trimethylpentanediol-1,5,2,2-dimethylpropanediol-1,3,1,4-dimethylolcyclohexane, 1,6-dimethylolcyclohexane, glycerol, trimethylolpropane, erythritol, pentaerythritol, sorbitol etc. The compounds A) are then, for example, hydroxy methyl (meth)acrylate, hydroxyethyl ethacrylate, 2-hydroxyethyl (meth)acrylate, 2-hydroxypropyl (meth)acrylate; 3-hydroxypropyl (meth)acrylate, 3-hydroxybutyl (meth)acrylate, 4-hydroxybutyl (meth)acrylate, 6-hydroxyhexyl (meth)acrylate, 3-hydroxy-2-ethylhexyl (meth)acrylate, and di(meth)acrylic esters of 1,1,1-trimethylolpropane or of glycerol. Suitable compounds A) are also the esters and amides of the abovementioned α,β-ethylenically unsaturated mono- and dicarboxylic acids with C₂-C₁₂-aminoalcohols which have a primary or secondary amino group. These include aminoalkyl acrylates and aminoalkyl methacrylates and N-monoalkyl derivates thereof which carry, for example, an N—C₁- to C₈-monoalkyl radical, such as aminomethyl (meth)acrylate, aminoethyl (meth)acrylate, N-methylaminomethyl (meth)acrylate, N-ethylaminomethyl (meth)acrylate, N-ethylaminoethyl (meth)acrylate, N-(n-propyl)aminomethyl (meth)acrylate, N-isopropylaminomethyl (meth)acrylate and preferably tert-butylaminoethyl acrylate and tert-butylaminoethyl methacrylate. These also include N-(hydroxy-C₁-C₁₂-alkyl)(meth)acrylamides, N-hydroxymethyl(meth)acrylamide, N-hydroxyethyl-(meth)acrylamide etc.

Suitable compounds A) are also the amides of the abovementioned α,β-ethylenically unsaturated mono- and dicarboxylic acids with di- and polyamines which have at least two primary or two secondary or one primary and one secondary amino group(s). These include, for example, the corresponding amides of acrylic acid and methacrylic acid, such as aminomethyl(meth)acrylamide, aminoethyl(meth)acrylamide, aminopropyl(meth)acrylamide, amino-n-butyl(meth)acrylamide, methylaminoethyl(meth)acrylamide, ethylaminoethyl(meth)acrylamide, methylaminopropyl(meth)acrylamide, ethylaminopropyl(meth)acrylamide or methylamino-n-butyl(meth)acrylamide.

Suitable compounds A) are also the reaction products of epoxide compounds which have at least one epoxide group with the abovementioned α,β-ethylenically unsaturated mono- and/or dicarboxylic acids and anhydrides thereof. Suitable epoxide compounds are, for example, glycidyl ethers' such as bisphenol A diglycidyl ether, resorcinol diglycidyl ether, 1,3-propanediol diglycidyl ether, 1,4-butanediol diglycidyl ether, 1,5-pentanediol diglycidyl ether or 1,6-hexanediol diglycidyl ether.

### Component B)

Component B) is customary aliphatic, cycloaliphatic and/or aromatic diisocyanates, such as tetramethylene diisocyanate, hexamethylene diisocyanate, methylenediphenyl diisocyanate, 2,4- and 2,6-tolylene diisocyanate and isomer mixtures thereof, o- and m-xylylene diisocyanate, 1,5-naphthylene diisocyanate, 1,4-cyclohexylene diisocyanate, dicyclohexylmethane diisocyanate and mixtures thereof. Component B) is preferably hexamethylene diisocyanate, isophorone diisocyanate, o- and m-xylylene diisocyanate, dicyclohexylmethane diisocyanate and mixtures thereof. If desired, up to 3 mol % of said compounds can be replaced by triisocyanates.

### Component C)

Suitable compounds C) are, for example, diols, diamines, aminoalcohols, and mixtures thereof. The molecular weight of these compounds is preferably in a range from about 56 to 280. If desired, up to 3 mol % of said compounds can be replaced by triols or triamines.

Suitable diols C) are, for example, ethylene glycol, propylene glycol, butylene glycol, neopentyl glycol, cyclohexanedimethylol, di-, tri-, tetra-, penta- or hexaethylene glycol and mixtures thereof. Preference is given to using neopentyl glycol and/or cyclohexanedimethylol.

Suitable aminoalcohols, C) are, for example, 2-aminoethanol, 2-(N-methylamino)-ethanol, 3-aminopropanol, 4-aminobutanol, 1-ethylaminobutan-2-ol, 2-amino-2-methyl-1-propanol, 4-methyl-4-aminopentan-2-ol etc.

Suitable diamines C) are, for example, ethylenediamine, propylenediamine, 1,4-diaminobutane, 1,5-diaminopentane and 1,6-diaminohexane.

Preferred compounds C) are polymers with a number-average molecular weight in the range from about 300 to 5000, preferably about 0.400 to 4000, in particular 500 to 3000. These include, for example, polyesterdiols, polyetherols, α,ω-diamino polyethers and mixtures thereof. Preference is given to using polymers containing ether groups.

The polyetherols C) are preferably polyalkylene glycols, e.g. polyethylene glycols, polypropylene glycols, polytetrahydrofurans etc., block copolymers of ethylene oxide and propylene oxide or block copolymers of ethylene oxide, propylene oxide and butylene oxide which comprise the copolymerized alkylene oxide units in random distribution or in the form of blocks.

Suitable α,ω-diamino polyethers C) can be prepared, for example, by amination of polyalkylene oxides with ammonia.

Suitable polytetrahydrofurans C) can be prepared by cationic polymerization of tetrahydrofuran in the presence of acidic catalysts, such as, for example, sulfuric acid or fluorosulfuric acid. Such preparation processes are known to the person skilled in the art.

Polyesterdiols C) which can be used preferably have a number-average molecular weight in the range from about 400 to 5000, preferably 500 to 3000, in particular 600 to 2000.

Suitable polyesterdiols are all those which are usually used for producing polyurethanes, in particular those, based on aromatic dicarboxylic acids, such as terephthalic acid, isophthalic acid, phthalic acid, Na or K sulfoisophthalic acid etc., aliphatic dicarboxylic acids, such, as adipic acid or succinic acid etc., and cycloaliphatic dicarboxylic acids, such as 1,2-, 1,3- or 1,4-cyclohexanedicarboxylic acid. Suitable diols are, in particular, aliphatic diols, such as ethylene glycol, propylene glycol, 1,6-hexanediol, neopentyl glycol, diethylene glycol, polyethylene glycols, polypropylene glycols, 1,4-dimethylolcyclohexane, and poly(meth)acrylate diols of the formula

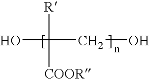

in which R′ is H or CH₃ and R″ is C₁-C₁₈-alkyl (in particular C₁-C₁₂- or C₁-C₈-alkyl), which have a molar mass of up to about 3000. Such diols can be produced in the usual way and are commercially available (Tegomer® grades MD, BD and OD from Goldschmidt).

Preference is given to polyesterdiols based on aromatic and aliphatic dicarboxylic acids and aliphatic diols, in particular those in which the aromatic dicarboxylic acid constitutes 10 to 95 mol %, in particular 40 to 90 mol % and preferably 50 to 85 mol %, of the total dicarboxylic acid fraction (remainder aliphatic dicarboxylic acids). Particularly preferred polyesterdiols are the reaction products of phthalic acid/diethylene glycol, isophthalic acid/1,4-butanediol, isophthalic acid/adipic acid/1,6-hexanediol, 5-NaSO₃-isophthalic acid/phthalic acid/adipic acid/1,6-hexanediol, adipic acid/ethylene glycol, isophthalic acid/adipic acid/neopentyl glycol, isophthalic acid/adipic acid/neopentyl glycol/diethylene glycol/dimethylolcyclohexane and 5-NaSO₃-isophthalic acid/isophthalic acid/adipic acid/neopentyl glycol/diethylene glycol/dimethylolcyclohexane.

The compounds C) can be used individually or as mixtures. Further possible constituents of these urethane (meth)acrylates are to be found in DE-A 198 38 852 p. 5, I. 40 to p. 9, I. 20, to the entire contents of which reference is hereby made.

As suitable component iii1), further mention may be made of:

iii1a) reaction products of the reaction of hydroxy(meth)acrylates with diols and/or OH-terminated polyols and/or OH-terminated polyesters and/or diamines and diisocyanates. Such difunctional urethane-acrylate oligomers and their preparation are described, for example, in WO 97/00664, p. 5, I. 17 to p 6, I. 8 and the corresponding examples, to the entire contents of which reference is hereby made.

iii1b) Carbamoyloxycarboxylates of the general formula I

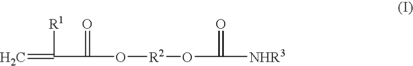

where
R¹ is H, halogen or C1-C8-alkyl,
R² is optionally substituted C₁-C₁₂-alkylene, -arylene, -alkylarylene or -arylalkylene, polyoxyalkylene,
R³ is C₁-C₈-alkyl.

Such carbamoyloxycarboxylates of the general formula I are disclosed in U.S. Pat. No. 3,479,328 and U.S. Pat. No. 3,674,838, to the entire contents of which reference is hereby made.

iii1c) The divinylurethanes of the general formula II disclosed in GB 1 443 715

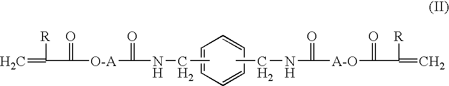

where
R is H or methyl
A is (poly)alkyleneoxy
and vinylurethanes of the general formula III

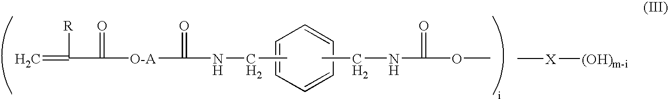

where R and A are as defined in formula II and X and n are as defined in GB 1 443 715 p. 2, I. 9-13. The vinylurethanes likewise described in GB 1 443 715, to the entire contents of which reference is hereby made, are further possible components c) of the polymers according to the invention.
iii1d) The N-substituted carbamoyloxyalkyleneoxyalkyl (meth)acrylates of the general formula IV described in EP-A 0 036 813, to the entire contents of which reference is hereby made,

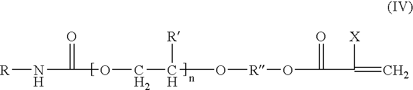

where R, R′, R″ and X are as defined in EP-A 0 036 813, p. 2, I, 13-28, and n is an integer from 0 to 20, preferably from 1 to 6 and particularly preferably from 1 to 4.

iii1e) The urethane acrylate compounds known from DE-A-4 007 146, to the entire contents of which reference is hereby made, which are obtainable by reacting polyisocyanates with hydroxylalkyl acrylates, followed by a reaction with primary or secondary amines.

iii1f) Products of the reaction of isocyanates with polyols and hydroxyalkyl acrylates, as described, for example, in DE 27 26 041 A, U.S. Pat. No. 4,260,703 and U.S. Pat. No. 4,481,093, and products of the reaction of isocyanates with hydroxyalkyl acrylates, as described in JP 63297369 and JP 59157112, to the entire contents of which reference is hereby made.

iii1g) The prepolymers comprising urethane groups described in EP-A 0 903 363, to the entire contents of which reference is hereby made, which are preparable by a method in which a component A comprising isocyanate groups is reacted with a component B comprising OH groups, where the component A comprises at least one trifunctional isocyanate compound A1 and, if appropriate, one or more difunctional isocyanate compounds A2, and the component B comprising OH groups comprises at least one olefinically unsaturated compound B1 with at least one reactive OH group and, if appropriate, compounds B2 comprising OH groups different therefrom, where either the component A comprises two different isocyanate compounds A1 or an isocyanate compound A1 and at least one isocyanate compound A2, or the component B comprises at least two different compounds B2.

iii1h) Polyurethane polymers which comprise A) 40 to 80% by weight, based on the total weight of components A) to F), of at least one prepolymer containing hydroxyl groups and having at least one free-radically or photochemically polymerizable α,β-ethylenically unsaturated double bond, where the prepolymer comprises, in copolymerized form,

A) a reaction product or a mixture of a) at least one polyester acrylate and/or polyether acrylate and/or polyurethane acrylate and b) at least one epoxy acrylate,
B) 0.1 to 20% by weight, based on the total weight of components A) to F), of at least one compound with at least one hydroxyl group reactive toward isocyanate groups and/or primary or secondary amino group and additionally at least one polar functional group,
C) 0.1 to 10% by weight, based on the total weight of components A) to F), of at least one compound chosen from diamines, polyamines and mixtures thereof,
D) 0 to 20% by weight, based on the total weight of components A) to F), of at least one further compound, different from A), B), C) and E), having at least two groups reactive toward isocyanate groups, which are hydroxyl groups and mixtures of hydroxyl groups and/or primary or secondary amino groups,
E) 0 to 20% by weight, based on the total weight of components A) to F), of at least one compound with a group which is reactive toward isocyanate groups,
F) 10 to 50% by weight, based on the total weight of components A) to F), of at least one polyisocyanate, and the salts thereof, wherein the sum of the hydroxyl numbers of components A) and D) is in a range from 121 to 300 mg of KOH/g.

These polyurethane polymers are described in EP-A-0 942 022, to the entire contents of which reference is hereby made.

iii1i) The reaction products, described in EP-A 1 002 818 (to the entire contents of which reference is hereby made), of

a) isocyanate trimers (trimer mixtures) based on aliphatic or cycloaliphatic diisocaynates which consist up to 100 mol % of compounds of the iminooxadiazinedione structure type of the formula A,

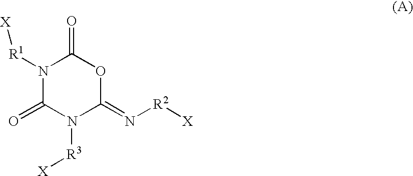

in which R¹, R² and R³, independently of one another, are optionally branched C4-C20-(cyclo)alkylene, and
X is identical or different radicals of isocyanate or of isocyanate secondary products which are of the iminooxadiazinedione, isocyanurate, uretdione, urethane, allophanate, biuret or oxadiazinetrione structure type and carry the abovementioned radicals R¹, R² and R³ in the N position, with
b) an alcohol component which comprises at least one monovalent hydroxy-functional optionally branched C₁-C₁₂-alkyl ester of (meth)acrylic acid.

iii1j) The polyurethanes comprising allyl groups of the general formula V, as disclosed in WO 01/72862-, to the entire contents of which reference is hereby made:

R¹—[NHCO(OR)_{y}(OCH₂CH═CH₂)ₘ]ₙ (V)

The meanings of R, R¹, y, m and n are described in WO 01172862 on p. 3, I. 29 to p. 4, I. 10.

iii1k) The (meth)acrylic-esters containing urethane groups described in WO 04/050888, to the entire contents of which reference is hereby made, which are preparable by reaction an alcohol containing urethane groups with (meth)acrylic acid or an ester of (meth)acrylic acid with a saturated alcohol and, if appropriate, purification of the reaction mixture, where the reaction is carried out in the presence of an enzyme (E).

iii1l) The urethane (meth)acrylate oligomers described in WO 98106783, to the entire contents of which reference is made at this point, in particular on p. 1, I. 22 to p. 2, I. 6.

iii1m) The polyurethanes described in DE 44 34 554 A1, to the entire contents of which reference is made at this point, in particular on p. 2, I. 42 to p. 4, I. 27.

iii1n) The urethane (meth)acrylate oligomers described in WO 04/067599, to the entire contents of which reference is hereby made, in particular on p. 10, I. 24 to p. 12, I. 13.

iii1o) The urethane acrylates described in U.S. Pat. No. 5,240,835, to the entire contents of which reference is made at this point, which are preparable by reacting alkyl acrylates with alcohols with catalysis of a biocatalyst of Corynebacterium oxydans.

iii1p) The carbamoyloxy(meth)acrylates described in WO 041052843, to the entire contents of which reference is made at this point, which are preparable by a method as is described on p. 3, I. 34 to p. 10, I. 28 of WO 04/052843.

iii1q) The carbamoyloxy(meth)acrylates which are described in WO 94/25537 p. 8, I. 29 to p. 9, I. 32, to the entire contents of which reference is hereby made.

iii1y) The polyisocyanate secondary products described in DE-A 102 46 112, to the entire contents of which reference is hereby made, comprising at least one allophanate group which carries at least one acrylate, methacrylate or vinyl ether double bond on the oxygen atom of the allophanate group bonded via two single bonds, wherein a polyisocyanate or polyisocyanate secondary product comprising at least one oxadiazinetrione group reacts with an alcohol comprising acryltate, methacrylate or vinyl ether double bond at temperatures from −20 to 100° C.

iii1r) WO 00/39183, to the entire contents of which reference is hereby made, describes compounds with isocyanate groups or capped isocyanate groups, allophanate groups and free-radically polymerizable C—C double bonds, where the C—C double bonds are activated by a carbonyl group directly bonded thereto or an O atom in ether function (activated double bonds), derived from polyisocyanates and alcohols A which also carry an activated double bond besides the alcohol group.

These compounds are preferably reacted with alcohols ROH which carry only one OH group or with amines RNH₂ or RR′NH in at least an amount which is sufficient to convert all isocyanate groups and capped isocyanate groups into urethane or urea groups.

Here, R and R′, independently of one another, are C₁-C₁₂-alkyl, -aryl, -alkylaryl or -arylalkyl, polyoxyalkylene, where the radicals may, if appropriate, be functionalized with hydroxyl groups.

Preferred alcohols for this reaction are C₁-C₁₂-, in particular C₁-C₄-alkanols, such as, for example, methanol, ethanol, n-propanol, isopropanol, n-butanol, isobutanol, sec-butanol, tert-butanol.

Preferred amines for this reaction are C₁-C₁₂-, in particular C₁-C₄-(di)alkylamines, (di)alkanolamines, alkylalkanolamines, such as, for example, ethylamine, butylamine, diethylamine, ethanolamine, diethanolamine, 2-amino-2-methylpropanol.

In this way, compounds of the following general formulae, for example, are obtained: From the reaction with alcohols:

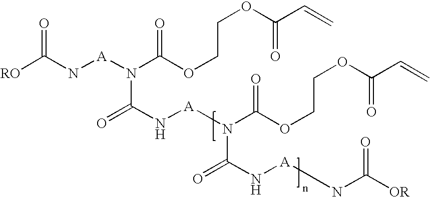

From the reaction with amines:

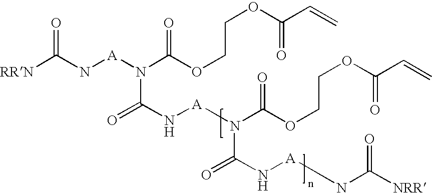

where
R is C₁-C₁₂-alkyl, -aryl, -alkylaryl or -arylalkyl, polyoxyalkylene, if appropriate functionalized with hydroxyl groups
R′ is H, C₁-C₁₂-alkyl, -aryl, -alkylaryl or -arylalkyl, polyoxyalkylene, if appropriate functionalized with hydroxyl groups
n is 0 to 10, preferably 0 to 5, particularly preferably 0 to 2
A is C₁-C₁₂-alkylene, -arylene, -alkylarylene or -arylalkylene, polyoxyalkylene and mixtures thereof.

The corresponding methacrylate derivatives of these compounds can of course also be used as component cl.

Suitable components c) are, for example, also

iii1s) N-butyl-2-hydroxyethylcarbamates (CAS 63225-53-6) of the formula

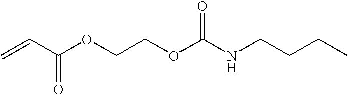

(as commercially available, for example, as Ebecryl®CL 1039 (UCB)) and the corresponding methacrylic acid derivative,

iii1t) N-Methyl-2-hydroxyethylcarbamates (CAS 52607-81-5) of the formula

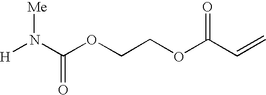

and the corresponding methyacrylic acid derivative,

iii1u) one of or a mixture or the two components of the following formulae (the mixture is referred to herein as monomer C22 (also see the examples)):

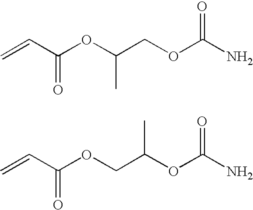

and the corresponding methacrylic acid derivatives,

iii1v) one of or a mixture of the two components of the following formulae

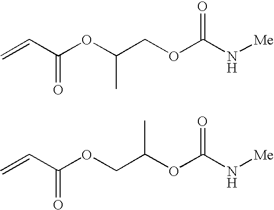

and the corresponding methacrylic acid derivatives,

iii1w) compound of the following formula

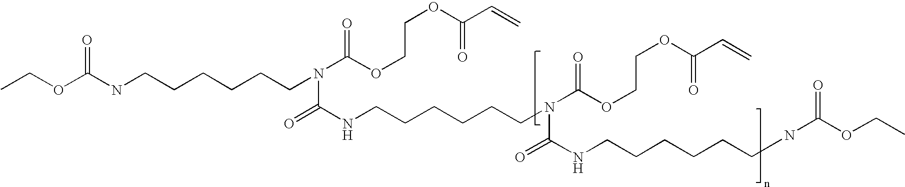

where n is 0 to 10, preferably 0 to 4, particularly preferably 0 to 2, and the corresponding methacrylic acid derivatives.

iii1x) Diurethane dimethacrylate 7,7,9- (or 7,9,9-)trimethyl-4,13-dioxo-3,14-dioxa-5,12-diazahexadecane-1,16-diol dimethacrylate (CAS 72869-86-4), which is commercially available, for example, as PLEX®36661-O (Degussa).

Polyurethane (meth)acrylates suitable as component iii1), such as polyurethane mono-, di-, tri-, tetra-, penta- or hexa(meth)acrylates are commercially available under the tradenames Laromer® (BASF), Photomer® (Cognis), Sartomer® (Sartomer) or Ebecryl® (UCB).

They can be used in pure form (without diluents), as solutions in solvents such as ethanol or butyl acetate or as solutions in reactive diluents (such as, for example, tripropylene glycol diacrylate (TPGDA), hexanediol diacrylate (HDDA), dipropylene glycol diacrylate (DPGDA), trimethylolpropane formal monoacrylate (Laromer®LR 8887), trimethylolpropane triacrylate (TMPTA), propoxylated glyceryl triacrylate (GPTA), ethoxylated trimethylolpropane triacrylate (EO3TMPTA), ethoxyethoxyethyl acrylate (EOEOEA), PEG 400 diacrylate (PEG400DA), isobornyl acrylate (IBOA), propoxylated neopentyl glycol diacrylate (PO2NPGDA), 2-phenoxyethyl acrylate (POEA), butanediol diacrylate (BDDA), butanediol acrylate (BDMA), dihydrodicyclopentadienyl acrylate (DCPA), triethylene glycol divinyl ether, ethyl digycol acrylate (EDGA), lauryl acrylate (LA), 4-t-butylcyclohexyl acrylate (TBCH), or as aqueous emulsions.

Such polyurethane (meth)acrylates are:

Laromer® grades LR 8949, LR 9005, LR 8983, UA 19 T, UA 9030V, UA 9028V, UA 9029V, UA 9033V, UA 9031V and LR 8987,
Photomer® grades 6891, 6892, 6893-20R, 6572, 6010, 6019, 6184, 6210, 6217, 6230, 6363 and 6008
Sartomer®CN grades, such as, for example,
the aliphatic urethane acrylates CN 934 CN 934X50, CN 944B85, CN 945A60, CN 945B85, CN 953B70, CN 961E75, CN 961H81, CN 962, CN 963A80, CN 963B80, CN 963E75, CN 963E80, CN 963J85, CN 964, CN 964A85, CN 964B85, CN 964H90, CN 964E75, CN 965, CN 965A80, CN 966A80, CN 966B85, CN 966H90, CN 966I80, CN 966J75, CN 966R⁶⁰, CN 968, CN 982E75, CN 982P90, CN 983, CN 983B88, CN 984, CN 985B88
and the aromatic urethane acrylates CN 970A60, CN 970E60, CN 970H75, CN 971A80, CN 972, CN 973A80, CN 973H85, CN 973J75, CN 975, CN 977C70, CN 978, CN 980, CN 980M50, CN 981, CN 981A75, CN 981B88, CN 982A75, CN 982B88 Ebecryl® grades, such as, for example, 220, 230, 244, 264, 265, 270.

As compound iii1), particular preference is given to carbamoyloxycarboxylates of the general formula VIII

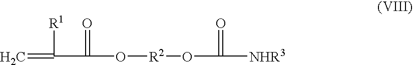

where
R¹ is H, halogen, C₁-C₈-alkyl, preferably H or methyl,
R² is optionally substituted C₁-C₁₂-alkylene, -arylene, -alkylarylene or -arylalkylene, optionally hydroxy-substituted polyoxyalkylene,
R³ is H, C₁-C₈-alkyl.

In general, preference is given to those compounds iii1) which comprise at most 4, preferably at most 3 and particularly preferably at most 2, free-radically polymerizable double bonds per molecule.

The polymers obtainable by the method according to the invention comprise 0-30% by weight, preferably 0.1-20% by weight, particularly preferably 0.5-10% by weight and most preferably 0.5-5% by weight, of the compound iii) in copolymerized form.

Component iii2)

Suitable components iii2) are the amides of (meth)acrylic acid different from iii3) and iii4). Such amides are, for example, (meth)acrylamide, N-methyl(meth)acrylamide, N-ethyl(meth)acrylamide, N-n-propyl(meth)acrylamide, N-isopropyl(meth)acrylamide, N-(n-butyl)methacrylamide, N-(sec-butyl)methacrylamide, N-(tert-butyl)methacrylamide, N-(n-pentyl)(meth)acrylamide, N-(n-hexyl)(meth)acrylamide, N-(n-heptyl)-(meth)acrylamide, N-(n-octyl)(meth)acrylamide, N-(tert-octyl)(meth)acrylamide, N-(1,1,3,3-tetramethylbutyl)(meth)acrylamide, N-ethylhexyl(meth)acrylamide, N-(n-nonyl)(meth)acrylamide, N-(n-decyl)(meth)acrylamide, N-(n-undecyl)(meth)acrylamide, N-tridecyl(meth)acrylamide, N-myristyl(meth)acrylamide, N-pentadecyl(meth)acrylamide, N-palmityl(meth)acrylamide, N-heptadecyl(meth)acrylamide, N-nonadecyl(meth)acrylamide, N-arachinyl(meth)acrylamide, N-behenyl(meth)acrylamide, N-lignocerenyl(meth)acrylamide, N-cerotinyl(meth)acrylamide, N-melissinyl(meth)acrylamide, N-palmitoleinyl(meth)acrylamide, N-oleyl(meth)acrylamide, N-linoleyl(meth)acrylamide, N-linolenyl(meth)acrylamide, N-stearyl(meth)acrylamide, N-lauryl(meth)acrylamide. Suitable components iii2) are also 2-hydroxyethylacrylamide, 2-hydroxyethylmethacrylamide, 2-hydroxyethylethacrylamide, 2-hydroxypropylacrylamide, 2-hydroxypropylmethacrylamide, 3-hydroxypropylacrylamide, 3-hydroxypropylmethacrylamide, 3-hydroxybutylacrylamide, 3-hydroxybutylmethacrylamide, 4-hydroxybutylacrylamide, 4-hydroxybutylmethacrylamide, 6-hydroxyhexylacrylamide, 6-hydroxyhexylmethacrylamide, 3-hydroxy-2-ethylhexylacrylamide and 3-hydroxy-2-ethylhexylmethaciylamide.

Components iii3) and iii4)

The components iii3) and iii4) are monomers which comprise at least one cationogenic and/or cationic group per molecule.

The cationogenic and cationic groups are preferably nitrogen-containing groups, such as primary, secondary and tertiary amino groups, and quaternary ammonium groups.

The nitrogen-containing groups are preferably tertiary amino groups.

The components iii3) and iii4) are preferably used in noncharged form for the polymerization. However, use in charged form is also suitable. Charged cationic groups can be produced, for example, from the amine nitrogen atoms by protonation, for example with monobasic or polybasic carboxylic acids, such as lactic acid or tartaric acid, or mineral acids, such as phosphoric acid, sulfuric acid and hydrochloric acid.

Preferably, the components iii3) and iii4) are chosen from
esters of α,β-olefinically unsaturated mono- and dicarboxylic acids with aminoalcohols, which may be mono- or dialkylated on the amine nitrogen,
amides of α,β-olefinically unsaturated mono- and dicarboxylic acids with diamines which have at least one primary or secondary amino group,
N,N-diallylamine,
N,N-diallyl-N-alkylamines and derivatives thereof,
vinyl- and allyl-substituted nitrogen heterocycles,
vinyl- and allyl-substituted heteroaromatic compounds and
mixtures thereof.

Suitable components iii3) and iii4) are also the esters of α,β-olefinically unsaturated mono- and dicarboxylic acids with aminoalcohols. Preferred aminoalcohols are C₂-C₁₂-aminoalcohols which are C₁-C₈-mono- or -dialkylated on the amine nitrogen. Suitable as acid component of these esters are, for example, acrylic acid, methacrylic acid, fumaric acid, maleic acid, itaconic acid, crotonic acid, maleic anhydride, monobutyl maleate and mixtures thereof. Preference is given to using acrylic acid, methacrylic acid and mixtures thereof.

Particularly preferred components iii3) and iii4) are N-methylaminoethyl (meth)acrylate, N-ethylaminoethyl (meth)acrylate, N-(n-propyl)aminoethyl (meth)acrylate, N-(n-butyl)aminoethyl (meth)acrylate, N-(tert-butyl)aminoethyl (meth)acrylate, N,N-dimethylaminomethyl (meth)acrylate, N,N-dimethylaminoethyl (meth)acrylate, N,N-diethylaminoethyl (meth)acrylate, N,N-dimethylaminopropyl (meth)acrylate, N,N-diethylaminopropyl (meth)acrylate and N,N-dimethylaminocyclohexyl (meth)acrylate.

In particular, N-(tert-butyl)aminoethyl acrylate and N-(tert-butyl)aminoethyl methacrylate are used as components iii3) and iii4).

Suitable components iii3) and iii4) are also the amides of the abovementioned α,β-olefinically unsaturated mono- and dicarboxylic acids with diamines which have at least one primary or secondary amino group.

Preference is given to diamines which have one tertiary and one primary or secondary amino group. As components iii3) and iii4), preference is given to using N-[2-(dimethylamino)ethyl]acrylamide, N-[2-(dimethylamino)ethyl]methacrylamide, N-[3-(dimethylamino)propyl]acrylamide, N-[3-(dimethylamino)propyl]methacrylamide, N-[4-(dimethylamino)butyl]acrylamide, N-[4-(dimethylamino)butyl]methacrylamide, N-[2-(diethylamino)ethyl]acrylamide, N-[4-(dimethylamino)cyclohexyl]acrylamide and N-[4-(dimethylamino)cyclohexyl]methacrylamide.

Particular preference is given to using N-[3-(dimethylamino)propyl]acrylamide and/or N-[3-(dimethylamino)propyl]methacrylamide.

Suitable components iii3) and iii4) are also N,N-diallylamines and N,N-diallyl-N-alkylamines and acid addition salts thereof. Alkyl here is preferably C₁-C₂₄-alkyl. Preference is given to N,N-diallyl-N-methylamine.

Suitable components iii3) and iii4) are also vinyl- and allyl-substituted nitrogen heterocycles, such as N-vinylimidazole, N-vinylimidazole derivatives, e.g.: N-vinyl-2-methylimidazole, vinyl- and allyl-substituted heteroaromatic compounds, such as 2- and 4-vinylpyridine, 2- and 4-allylpyridine, and the salts thereof.

Suitable components iii3) and iii4) are also N-vinylimidazoles of the general formula VIII, in which R¹ to R³ is hydrogen, C₁-C₄-alkyl or phenyl

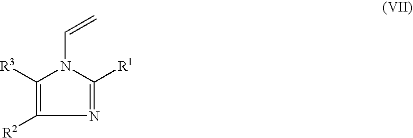

Examples of compounds of the general formula VII are to be found in table 1 below:

TABLE 1

| R¹: | R²: | R³: |
|---|---|---|
| H | H | H |
| Me | H | H |
| H | Me | H |
| H | H | Me |
| Me | Me | H |
| H | Me | Me |
| Me | H | Me |
| Ph | H | H |
| H | Ph | H |
| H | H | Ph |
| Ph | Me | H |
| Ph | H | Me |
| Me | Ph | H |
| H | Ph | Me |
| H | Me | Ph |
| Me | H | Ph |

Me = methyl;
Ph = phenyl

The components iii3) and iii4) are particularly preferably chosen from N-(tert-butylamino)ethyl (methyacrylate, N,N-dimethylaminoethyl (meth)acrylate, N-[3-(dimethylamino)propyl](meth)acrylamide, vinylimidazole and mixtures thereof.

If the polymers produced by the method according to the invention comprise compounds iii3) and/or iii4) in copolymerized form, then they comprise at least 0.1% by weight, preferably at least 1% by weight, particularly preferably at least 2% by weight and in particular at least 3% by weight and at most 30% by weight, preferably at most 20% by weight, particularly preferably at most 15% by weight and especially at most 10% by weight, of the components iii3) and/or iii4), based on the total weight of the compounds i) to iii) used.

The charged cationic groups can be produced from the amine nitrogens by quaternization with so-called alkylating agents. Examples of suitable alkylating agents are C₁-C₄-alkyl halides, or sulfates, such as ethyl chloride, ethyl bromide, methyl chloride, methyl bromide, dimethyl sulfate and diethyl sulfate. A quaternization can generally take place before or after the polymerization.

Component iii5)

Component iii5) are compounds with at least 2 free-radically polymerizable nonconjugated double bonds per molecule.

Suitable components iii5) are, for example, acrylic esters, methacrylic esters, allyl ethers or vinyl ethers of at least dihydric alcohols. The OH groups of the parent alcohols may here be completely or partially etherified or esterified; however, the components iii5) comprise at least two free-radically polymerizable unsaturated groups. Examples of the parent alcohols are dihydric alcohols, such as 1,2-ethanediol, 1,2-propanediol, 1,3-propanediol, 1,2-butanediol, 1,3-butanediol, 2,3-butanediol, 1,4-butanediol, but-2-ene-1,4-diol, 1,2-pentanediol, 1,5-pentanediol, 1,2-hexanediol, 1,6-hexanediol, 1,10-decanediol, 1,2-dodecanediol, 1,2-dodecanediol, neopentyl glycol, 3-methylpentane-1,5-diol, 2,5-dimethyl-1,3-hexanediol, 2,2,4-trimethyl-1,3-pentanediol, 1,2-cyclohexanediol, 1,4-cyclohexanediol, 1,4-bis(hydroxymethyl)cyclohexane, hydroxypivalic neopentyl glycol monoester, 2,2-bis(4-hydroxyphenyl)propane, 2,2-bis[4-(2-hydroxypropyl)phenyl]propane, diethylene glycol, triethylene glycol, tetraethylene glycol, dipropylene glycol, tripropylene glycol, tetrapropylene glycol, 3-thiopentane-1,5-diol, and polyethylene glycols, polypropylene glycols and polytetrahydrofurans with molecular weights of in each case 200 to 10 000.

Apart from the homopolymers of ethylene oxide or propylene oxide, it is also possible to use block copolymers of ethylene oxide or propylene oxide or copolymers which comprise ethylene oxide and propylene oxide groups in incorporated form.

Examples of parent alcohols having more than two OH groups are trimethylolpropane, glycerol, pentaerythritol, 1,2,5-pentanetriol, 1,2,6-hexanetriol, triethoxycyanuric acid, sorbitan, sugars such as sucrose, glucose, mannose. Preferred polyhydric alcohols in this connection are also di- and trisaccharides.

The polyhydric alcohols can of course also be used following reaction with ethylene oxide or propylene oxide as the corresponding ethoxylates or propoxylates, respectively. The polyhydric alcohols can also firstly be converted into the corresponding glycidyl ethers by reaction with epichlorohydrin.

Further suitable components iii5) are the vinyl esters or the esters of monohydric, unsaturated alcohols with olefinically unsaturated C₃-C₈-carboxylic acids, for example acrylic acid, methacrylic acid, itaconic acid, maleic acid or fumaric acid. Examples of such alcohols are allyl alcohol, 1-buten-3-ol, 5-hexen-1-ol, 1-octen-3-ol, 9-decen-1-ol, dicyclopentenyl alcohol, 10-undecen-1-ol, cinnamyl alcohol, citronellol, crotyl alcohol or cis-9-octadecen-1-ol. The monohydric, unsaturated alcohols can, however, also be esterified with polybasic carboxylic acids, for example malonic-acid, tartaric acid, trimellitic acid, phthalic acid, terephthalic acid, citric acid or succinic acid.

Further suitable components iii5) are esters of unsaturated carboxylic acids with the above-described polyhydric alcohols, for example oleic acid, crotonic acid, cinnamic acid or 10-undecenoic acid.

Suitable components iii5) are, furthermore, straight-chain or branched, linear or cyclic, aliphatic or aromatic hydrocarbons which have at least two double bonds which, in the case of aliphatic hydrocarbons, must not be conjugated, e.g. divinylbenzene, divinyltoluene, 1,7-octadiene, 1,9-decadiene, 4-vinyl-1-cyclohexene, trivinylcyclohexane or polybutadienes with molecular weights of from 200 to 20 000. Also suitable as components iii5) are the amides of (meth)acrylic acid, itaconic acid and maleic acid, and N-allylamines of at least difunctional amines. Such amines are, for example, 1,2-diaminomethane, 1,2-diaminoethane, 1,3-diaminopropane, 1,4-diaminobutane, 1,6-diaminohexane, 1,12-dodecanediamine, piperazine, diethylenetriamine or isophoronediamine. Likewise suitable are the amides of allylamine and unsaturated carboxylic acids such as acrylic acid, methacrylic acid, itaconic acid, maleic acid, or at least dibasic carboxylic acids, as have been described above. Also suitable as component iii5) are triallylamine and triallylmonoalkyl-ammonium salts, e.g. triallylmethylammonium chloride or methylsulfate. Also suitable are N-vinyl compounds of urea derivatives, at least difunctional amides, cyanurates or urethanes, for example of urea, ethyleneurea, propyleneurea or tartardiamide, e.g. N,N′-divinylethyleneurea or N,N′-divinylpropyleneurea. Also suitable are alkylenebisacrylamides, such as methylenebisacrylamide and N,N′-(2,2)butane and 1,1′-bis(3,3′-vinylbenzimidazolith-2-one)-1,4-butane. Other suitable components iii5) are, for example, alkylene glycol di(meth)acrylates, such as ethylene glycol diacrylate, ethylene glycol dimethacrylate, tetraethylene glycol acrylate, tetraethylene glycol dimethacrylate, dimethylene glycol acrylate, diethylene glycol methacrylate, vinyl acrylate, allyl acrylate, allyl methacrylate, divinyldioxane, pentaerythritol allyl ether, and mixtures of these components iii5).

Further suitable components iii5) are divinyldioxane, tetraallylsilane or tetravinylsilane. Particularly preferably used components iii5) are, for example, methylenebisacrylamide, triallylamine and triallylalkylammonium salts, divinylimidazole, pentaerythritol triallyl ether, N,N′-divinylethyleneurea, reaction products of polyhydric alcohols with acrylic acid or methacrylic acid, methacrylic esters and acrylic esters of polyalkylene oxides or polyhydric alcohols which have been reacted with ethylene oxide and/or propylene oxide and/or epichlorohydrin.

Very particularly preferred as component iii5) are pentaerythritol triallyl ether, methylenebisacrylamide, N,N′-divinylethyleneurea, triallylamine and triallylmonoalkylammonium salts, and acrylic esters of glycol, butanediol, trimethylolpropane or glycerol or acrylic esters of glycol, butanediol, trimethylolpropane or glycerol reacted with ethylene oxide and/or epichlorohydrin.

It is of course also possible to use mixtures of the abovementioned compounds. The component iii5) is preferably soluble in the reaction medium. If the solubility of component iii5) in the reaction medium is low, then it can be dissolved in a monomer or in a monomer mixture, or else be metered in dissolved form in a solvent which is miscible with the reaction medium. Particular preference is given to those components iii5) which are soluble in the monomer mixture.

If component iii5) is used for producing the polymers, then the amount used is at least 0.01% by weight, preferably at least 0.05% by weight, particularly preferably at least 0.1% by weight and at most 5%, by weight, preferably at most 2% by weight and EP-A 0 686 621, to the entire contents of which reference is hereby made, also describes suitable components iii7). These are reaction products of (meth)acrylic acid with a hydroxy compound. Suitable hydroxy compounds are compounds with one or more hydroxy groups. Monoalcohols, C₂-C₆-alkylenediols, trimethylolpropane, glycerol or pentaerythritol or compounds comprising hydroxy groups and alkoxylated, for example with ethylene oxide or propylene oxide are specified.

Suitable hydroxy compounds are also polyesters containing hydroxyl groups. Such polyesters containing hydroxyl groups may be produced, for example, in the customary way by esterification of dicarboxylic acids or polycarboxylic acids with diols or polyols. The starting materials for such polyesters containing hydroxyl groups are known to the person skilled in the art. Dicarboxylic acids which can preferably be used are succinic acid, glutaric acid, adipic acid, sebacic acid, o-phthalic acid, their isomers and hydrogenation products, and esterifiable derivatives, such as anhydrides, e.g. maleic anhydride, or dialkyl esters of said acids. Suitable as polycarboxylic acid is, for example, trimellitic acid. The polyesterols to be used also include polycaprolactonediols and -triols, the preparation of which is likewise known to the person skilled in the art. Preferred hydroxy compounds are saturated polyesters comprising at least 2, in particular 2 to 6, free hydroxyl groups which may, if appropriate, also comprise ether groups, or polyethers (as component iii8)) with at least 2, in particular 2 to 6, free hydroxyl groups.

The components iii7), such as, for example, polyester (meth)acrylates, have at least 2 free-radically polymerizable double bonds per molecule. It is also preferred to use mixtures of components iii), for example of polyester (meth)acrylates which comprise on average more than 2 free-radically polymerizable, olefinically unsaturated double bonds per molecule of polyester (meth)acrylate. Such mixtures are produced, for example, by mixing compounds having in each case 2 and compounds having in each case 3 or more polymerizable double bonds per molecule. Compounds which comprise only one or no double bond per molecule may of course also be present in the mixtures. Such compounds are then, however, present in amounts such that the average number of polymerizable double bonds per molecule is nevertheless more than 2.

Components iii8)

The term polyether is known to the person skilled in the art. Polyethers are polymers whose organic repeat units are held together by ether functionalities (C—O—C). Examples of polyethers are polyalkylene glycols (polyethylene glycols, polypropylene glycols, polyepichlorohydrins) as polymers of 1,2-epoxides, epoxide resins, polytetrahydrofurans (polytetramethylene glycols), polyoxetanes, polyphenylene ethers (polyaryl ethers) or polyether (ether) ketone (ketone)s.

Components iii8) according to this invention are, for example, polyether (meth)acrylates which comprise at least 2 free-radically polymerizable double bonds per molecule. These are likewise known in principle to the person skilled in the art.

They can be prepared by various methods. For example, polyethers containing hydroxyl groups, which are esterified with acrylic acid and/or methacrylic acid to give the polyether (meth)acrylates, can be obtained by reacting di- and/or polyhydric alcohols with various amounts of ethylene oxide and/or propylene oxide by well known methods (cf. e.g. Houben-Weyl, volume XIV, 2, Makromolekulare Stoffe II (Macromolecular Substances II), (1963)). It is also possible to use polymerization products of tetrahydrofuran or butylene oxide.

DE 2 853.921, to the entire contents of which reference is hereby made, also describes suitable components iii8), such as, for example, aliphatic or aromatic-aliphatic polyethers which are obtained by reacting di- and/or polyhydric alcohols with various amounts of ethylene oxide and/or propylene oxide and whose free hydroxyl groups are completely or partially etherified with ethylenically unsaturated alcohols, for example allyl alcohol, methallyl alcohol, crotyl alcohol, cinnamyl alcohol, and/or are esterified with α,β-ethylenically unsaturated monocarboxylic acids.

Polyether acrylates suitable as component iii8) are described, for example, in EP-A 0 279 303, to the entire contents of which reference is hereby made.

These polyether acrylates are obtainable by reacting A) 1 equivalent of a 2- to 6-hydric oxyalkylated C₂- to C₁₀-alcohol with B) 0.05 to 1 equivalent of a 2- to 4-basic Cr to C₁₀-carboxylic acid or anhydrides thereof and C) 0.1 to 1.5 equivalents of acrylic acid and/or methacrylic acid, and reacting the excess carboxyl groups with the equivalent amount of an epoxide compound.

EP-A 0 686 621, to the entire contents of which reference is hereby made, also describes suitable components iii8). These are reaction products of (meth)acrylic acid with a hydroxy compound. Suitable hydroxy compounds are compounds with one or more hydroxy groups. Compounds comprising hydroxy groups and alkoxylated, for example, with ethylene oxide or propylene oxide may be specified.

Preferred hydroxy compounds are saturated polyethers with at least 2, in particular 2 to 6, free hydroxyl groups. Suitable polyethers containing hydroxyl groups are, for example, those which can be obtained by known methods by reacting di- and/or polyhydric alcohols with various amounts of ethylene oxide and/or propylene oxide. In the case of the ethylene glycol/propylene glycol mixed condensation products, the reaction can expediently be controlled such that predominantly primary hydroxyl groups arise in terminal positions. Polymerization products of tetrahydrofuran or butylene oxide which comprise hydroxyl groups can also likewise be used.

Examples of component iii8) are polyalkylene glycol (meth)acrylates.

In a preferred embodiment of the invention, the compounds used as component iii8) are those whose molecular weight M_{w} is at least 200 g/mol, particularly preferably at least 400 g/mol, very particularly preferably at least 500 g/mol and most preferably more than 700 g/mol.

In a further preferred embodiment of the invention, compounds iii7) and/or iii8) or mixtures of compounds iii7) and/or iii8), where the average number of olefinic, free-radically polymerizable double bonds per molecule is more than 2, are used as particularly preferably at most 1% by weight, based on the total amount of components i) to iii).

If the polymers are to comprise a component iii5) in copolymerized form, then it is particularly advantageous to use mixtures of components iii1) and iii5). Such mixtures are commercially available and, besides component iii), usually comprise substances referred to as reactive diluents, such as, for example, tripropylene glycol diacrylate (TPGDA), hexanediol diacrylate (HDDA), dipropylene glycol diacrylate (DPGDA), trimethylolpropane formal monoacrylate (Laromer®LR 8887), trimethylolpropane triacrylate (TMPTA), propoxylated glyceryl triacrylate (GPTA), ethoxylated trimethylolpropane triacrylate (EO3TMPTA), ethoxyethoxyethyl acrylate (EOEOEA), PEG 400 diacrylate (PEG400DA), isobornyl acrylate (IBOA), propoxylated neopentyl glycol diacrylate (PO2NPGDA), 2-phenoxyethyl acrylate (POEA), butanediol diacrylate (BDDA), butanediol acrylate (BDMA), dihydrodicyclopentadienyl acrylate (DCPA), triethylene glycol divinyl ether, ethyl diglycol acrylate (EDGA), lauryl acrylate (LA), 4-t-butylcyclohexyl acrylate (TBCH).

Components iii6)

The compounds iii6) containing amide groups different from iii2) to iii4) are preferably chosen from compounds of the general formula VI

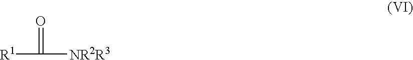

where R¹ is a group of the formula CH₂═CR⁴— where R⁴=H or C₁-C₄-alkyl and R² and R³, independently of one another, are H, alkyl, cycloalkyl, heterocycloalkyl, aryl or hetaryl, or R² and R³ together with the nitrogen atom to which they are bonded, are a five- to eight-membered nitrogen heterocycle or

R² is a group of the formula CH₂═CR⁴— and R¹ and R³, independently of one another, are H, alkyl, cycloalkyl, heterocycloalkyl, aryl or hetaryl, or R¹ and R³, together with the amide group to which they are bonded, are a lactam having 5 to 8 ring atoms. N-Vinyllactams are preferred as component iii6). Suitable as component iii6) are unsubstituted N-vinyllactams and N-vinyllactam derivatives which can have, for example, one or more C₁-C₆-alkyl substituents, such as methyl, ethyl, n-propyl, isopropyl, n-butyl, sec-butyl, tert-butyl etc. These include, for example, N-vinylpyrrolidone, N-vinylpiperidone, N-vinylcaprolactam, N-vinyl-5-methyl-2-pyrrolidone, N-vinyl-5-ethyl-2-pyrrolidone, N-vinyl-6-methyl-2-piperidone, N-vinyl-6-ethyl-2-piperidone, N-vinyl-7-methyl-2-caprolactam, N-vinyl-7-ethyl-2-caprolactam etc., and mixtures thereof.

Preferred components iii6) are those for which, in formula VI, R² is CH₂═CH— and R¹ and R³, together with the amide group to which they are bonded, are a lactam with 5 ring atoms.

Particular preference is given to using N-vinylpyrrolidone, N-vinylcaprolactam, N-vinylformamide, acrylamide or mixtures thereof, with N-vinylpyrrolidone being most preferred.

Components iii7)

The term polyester is known to the person skilled in the art. Polyesters are polymers with ester bonds —[—CO—O—]— in the main chain. Components iii7) according to this invention are, for example, polyester (meth)acrylates which comprise at least two free-radically polymerizable, olefinically unsaturated double bonds per molecule. Polyester (meth)acrylates are known in principle to the person skilled in the art. They can be prepared by various methods. For example, (meth)acrylic acid can be used directly as acid component when constructing the polyesters. In addition, there is the possibility of using hydroxyalkyl esters of (meth)acrylic-acid as alcohol component directly when constructing the polyesters. Preferably, however, the polyester (meth)acrylates are prepared by (meth)acrylation of polyesters. For example, polyesters containing hydroxyl groups can firstly be constructed, which are then reacted with acrylic or methacrylic acid. Preference is given to reacting at least 2 of the hydroxyl groups per molecule of the polyester containing hydroxyl groups with (meth)acrylic acid; so that, per molecule of the reaction product, at least two free-radically polymerizable, olefinically unsaturated double bonds are present.

It is also possible to firstly construct polyesters containing carboxyl groups, which are then reacted with a hydroxyalkyl ester of acrylic or methacrylic acid. Here too, at least two of the carboxyl groups per molecule of the polyester containing carboxyl groups are reacted with the hydroxyalkyl ester of (meth)acrylic acid, meaning that, per molecule of the reaction product, at least two free-radically polymerizable, olefinically unsaturated double bonds are present.

It is preferred to use mixtures of polyester (meth)acrylates which comprise, on average, more than 2 free-radically polymerizable, olefinically unsaturated double bonds per molecule of polyester (meth)acrylate.

Polyester acrylates suitable as component iii7) are described, for example, in EP-A 0 279 303, to the entire contents of which reference is hereby made (EP-A 0 279,303, p. 5, I. 2844).

DE 2 853 921 also describes suitable polyester acrylates, namely those of aliphatic and/or aromatic dicarboxylic acids, such as succinic acid, glutaric acid, adipic acid, pimelic acid, suberic acid, azelaic acid, sebacic acid, cyclohexanedicarboxylic acid, phthalic acid, isophthalic acid, terephthalic acid, maleic acid, fumaric acid, itaconic acid or derivatives thereof and polyhydric alcohols, such as ethylene glycol, polyethylene glycols, propylene glycol, polypropylene glycols, butanediol, hexanediol, neopentyl glycol, hydroxypivalic neopentyl glycol ester, trimethylolpropane, glycerol, pentaerythritol and/or trishydroxyethyl isocyanurate, and α,β-ethylenically unsaturated monocarboxylic acids, for example acrylic acid, methacrylic acid, crotonic acid, cinnamic acid and/or dicarboxylic acid half-esters of monoalkanols, such as maleic, fumaric and itaconic acid half-esters with C₁-C₄-monoalcohols, where acrylic acid and methacrylic acid are preferred.

component iii). Such mixtures are produced, for example, by mixing compounds having in each case 2 and compounds having in each case 3 or more polymerizable double bonds per molecule. Compounds which comprise only one or no double bond per molecule may of course also be present in the mixtures. Such compounds are then, however, present in amounts such that the average number of polymerizable double bonds per molecule is nevertheless more than 2.

It should be emphasized at this point that there are compounds suitable as component iii) which can be assigned to both groups iii7) and iii8) since they comprise both ester groups and ether groups. According to the invention, component iii) are thus also compounds comprising at least two free-radically polymerizable, olefinically unsaturated double bonds which at the same time comprise both ether and ester structures.

Commercially available products which are suitable as component iii) are, for example: Photomer®5010, Photomer®5429, Photomer®5430, Photomer®5432, Photomer®5662, Photomer®5806, Photomer®5930 from Cognis;

the Resin® grades from UCB, such as, for example, Resin®80, 81, 83, 450, 657, 770, 809, 810, 830, 835, 870, 1657, 1810, 1870, 2047**, 2870;
the CN® grades from Sartomer, such as, for example, CN293, CN294, CN296, CN292, CN2297A, CN2279, CN2280, CN2470, CN295, CN2300, CN2200, CN2203, CN2282, CN2284, CN2270, CN2271, CN2272, CN2273, CN2276, CN2250, CN2251, CN2252, CN2253, CN2255, CN2256, CN2257, CN2258, CN2259, CN2260, CN2261;
AROPLAZ®4097-WG4-55 from Reichhold;
Syntholux®-PE grades from Syhthopol as polyester acrylates and the Syntholux®-PA grades from Synthopol as polyether acrylates;
Laromer® grades Laromer®PE 55F, Laromer®PE 56F, Laromer®PE 46T, Laromer®9004, Laromer®PE 44F, Laromer®8800, Laromer®LR 8981, Laromer®LR 8992, Laromer®PE 22WN, Laromer®PE 55WN, Laromer®PO 33F, Laromer®LR 8863, Laromer®PO 43F, Laromer®LR 8967, Laromer®LR 8982, Laromer®LR 9007 (BASF).

As component iii), it is also possible to use vinyl acetate, vinyl propionate, vinyl butyrate, ethylene, propylene, isobutylene, butadiene, styrene, α-methylstyrene, acrylonitrile, methacrylonitrile, vinyl chloride, vinylidene chloride, vinyl fluoride, vinylidene fluoride and mixtures thereof.

The components iii) may also be silicone-containing compounds, in particular also (poly)urethane acrylates comprising silicone groups.

The polymers obtainable by the method according to the invention comprise 0-30% by weight, preferably 0.1-20% by weight, particularly preferably 0.5-10% by weight and most preferably 0.5-5% by weight, of component iii) in copolymerized form.

### Solution Polymerization

According to the invention, the polymers are prepared by solution polymerization in a solution comprising alcohol, where the polymerization solution comprises water in the range from more than 25 to 50% by weight.

Suitable alcohols are, for example, methanol, ethanol, n-propanol, isopropanol, n-butanol, sec-butanol, tert-butanol, 3-methyl-1-butanol (isoamyl alcohol), n-hexanol, cyclohexanol or glycols, such as ethylene glycol, propylene glycol and butylene glycol, and alkyl ethers of polyhydric alcohols, such as diethylene glycol, triethylene glycol, polyethylene glycols with number-average molecular weights up to about 3000, glycerol and dioxane.

Particularly preferably, the alcohol is or comprises ethanol and/or isopropanol, in particular ethanol.

In addition to alcohol and water, further solvents may be present in the polymerization solution. Of suitability in principle are all those solvents which are suitable for the free-radical polymerization, such as, for example, acetone, acetonitrile, aniline, anisole, benzonitrile, tert-butyl methyl ether (TBME), gamma-butyrolactone, quinoline, chloroform, cyclohexane, diethyl ether, dimethylacetamide, dimethylformamide, dimethyl sulfoxide, dioxane, glacial acetic acid, acetic anhydride, ethyl acetate, ethylene dichloride, ethylene glycol dimethyl ether, formamide, hexane, methylene chloride, methyl ethyl ketone, N-methylformamide, petroleum-ether/light benzine, piperidine, propylene carbonate-(4-methyl-1,3-dioxol-2-one), sulfolane, tetrachloroethene, tetrachloromethane, tetrahydrofuran, toluene, 1,1,1-trichloroethane, trichloroethene, triethylene glycol dimethyl ether (triglyme).

Polymerization solution is understood as meaning the substance mixture which is present following the addition of all of the components and after the polymerization is complete and before the first work-up step, such as, for example, drying, neutralization or steam distillation.

The polymerization solution comprises more than 25 up to at most 50% by weight of water and preferably more than 20 to at most 60% by weight of alcohol, where the total amount of water and alcohol is preferably at most 95% by weight, particularly preferably at most 90% by weight and in particular at most 80% by weight, of the polymerization solution.

The amount of water is preferably in the range from more than 25 to 45% by weight, particularly preferably in the range from 28 to 40% by weight, based on the polymerization solution.

The amount of alcohol is preferably in the range from 25 to 45% by weight, particularly preferably in the range from 30 to 40% by weight, based on the polymerization solution.

The amount of substances further present in the polymerization solution, which are essentially the components i), ii), iii) and the initiator, is preferably at least 5% by weight, particularly preferably at least 10% by weight and in particular at least 20% by weight, of the polymerization solution. Very particularly preferably, this amount is in the range from 27 to 37% by weight and most preferably in the range from 29 to 35% by weight, based on the polymerization solution. This amount can also be referred to as solids content of the polymerization solution.

Preference is given to a method according to the invention in which the temperature at which the polymerization is carried out is in the range from 30° C. to 120° C., particularly preferably in the range from 40° C. to 100° C.

The polymerization is usually carried out under atmospheric pressure, although it can also proceed under reduced or increased pressure. A suitable pressure range is between 1 and 10 bar.

In one preferred embodiment of the method, the polymerization is carried out at a pressure in the range from 2 to 10 bar.

The initiator used for the free-radical polymerization is preferably at least one water-soluble polymerization initiator chosen from the group consisting of peroxides, hydroperoxides, peroxodisulfates, percarbonates, peroxide esters, azo compounds and mixtures thereof.

A water-soluble polymerization initiator is understood as meaning an initiator which is soluble at 20° C. and 1013 mbar to at least 1 g, preferably to at least 10 g, in 1 liter of water.

In one preferred embodiment of the invention, the water-soluble polymerization initiator is chosen from the group consisting of water-soluble azo compounds, hydrogen peroxide, lithium peroxodisulfate, sodium peroxodisulfate, potassium peroxodisulfate, ammonium peroxodisulfate and mixtures thereof.

In addition, the water-soluble polymerization initiator is preferably chosen from the group consisting of 2,2′-azobis[2-(5-methyl-2-imidazolin-2-yl)propane]dihydrochloride 2,2′-azobis[2-(2-imidazolin-2-yl)propane]dihydrochloride 2,2′-azobis[2-(2-imidazolin-2-yl)propane disulfate dihydrate 2,2′-azobis(2-methylpropionamide) dihydrochloride 2,2′-azobis[N-(2-carboxyethyl)-2-methylpropionamidine]tetrahydrate 2,2′-azobis[2-(3,4,5,6-tetrahydropyrimidin-2-yl)propane]dihydrochloride 2,2′-azobis{2-[1-(2-hydroxyethyl)-2-imidazolin-2-yl]propane}dihydrochloride 2,2-azobis[2-(2-imidazolin-2-yl)propane]2,2′-azobis{2-methyl-N-[1,1-bis(hydroxymethyl)-2-hydroxyethyl]propionamide 2,2′-azobis{2-methyl-N-[2-(1-hydroxybutyl)]propionamide}2,2′-azobis[2-methyl-N-(2-hydroxyethyl)propionamide] and mixtures thereof.

Water-soluble redox initiator systems may also be used as polymerization initiators.

Such redox initiator systems comprise at least one peroxide-containing compound in combination with a redox coinitiator e.g. reductive sulfur compounds, for example bisulfites, sulfites, thiosulfates, dithionites and tetrathionates of alkali metals and ammonium compounds. For example, combinations of peroxodisulfates with alkali metal or ammonium hydrogen sulfites can be used, e.g. ammonium peroxodisulfate and ammonium disulfite. The amount of peroxide-containing compound to redox coinitiator is in the range from 30:1 to 0.05:1.

Transition metal catalysts can additionally be used in combination with the initiators or the redox initiator systems, e.g. salts of iron, cobalt, nickel, copper, vanadium and manganese. Suitable salts are, for example, iron(II) sulfate, cobalt(II) chloride, nickel(II) sulfate, or copper(I) chloride. Based on the monomers, the reductive transition metal salt is used in a concentration of from 0.1 ppm to 1000 ppm. Thus, combinations of hydrogen peroxide with iron(II) salts can be used, such as, for example, 0.5 to 30% hydrogen peroxide and 0.1 to 500 ppm Mohr's salt.

In addition, redox coinitiators and/or transition metal catalysts can be co-used in combination with the abovementioned initiators, e.g. benzoin, dimethylaniline, ascorbic acid, and complexes of heavy metals, such as copper, cobalt, iron, manganese, nickel and chromium. The amounts of redox coinitiators and/or transition metal catalysts usually used are about 0.1 to 1000 ppm, based on the amounts of monomers used. Further suitable initiators are described in the chapters 20 and 21 of Macromolecules, Vol. 2, 2nd Ed., H. G. Elias, Plenum Press, 1984, New York, the entire contents of which are hereby incorporated by reference. Suitable photoinitiators are also described in S. P. Pappas, J. Rad. Cur., July 1987, p. 6, to the entire contents of which reference is hereby made.

The amount of the at least one water-soluble initiator used for the polymerization of the monomers is preferably from 0.0001 to 10% by weight, particularly preferably 0.001 to 5% by weight and in particular 0.02 to 3% by weight, based on the total amount of the monomers used.

To adjust the molecular weight, the polymerization can take place in the presence of at least one regulator. Regulators which can be used are the customary compounds known to the person skilled in the art, such as, for example, sulfur compounds, e.g. mercaptoethanol, 2-ethylhexyl thioglycolate, thioglycolic acid or dodecyl mercaptan, and tribromochloromethane or other compounds which have a regulating effect on the molecular weight of the polymers obtained. A preferred regulator is cysteine.

The solution polymerization can be carried out either as a batch process or in the form of a feed method, including monomer feed, stepped and gradient procedure. In general, preference is given to the feed method in which, if appropriate, some of the polymerization mixture is initially introduced and heated to the polymerization temperature, and then the remainder of the polymerization mixture is introduced into the polymerization zone usually via one or more, spatially separate feeds, continuously, stepwise or with overlap of a concentration gradient while maintaining the polymerization.

To achieve the purest possible polymers with a low-residual monomer content, the polymerization (main polymerization) may be followed by an after-polymerization step. The after-polymerization can take place in the presence of the same initiator system as or a different initiator system to the main polymerization. Preferably, the after-polymerization takes place at least at the same temperature as, preferably at a higher temperature than, the main polymerization. If desired, the reaction mixture can, after the polymerization or between the first and the second polymerization step, be subjected to stripping with steam or to steam distillation, which is particularly advantageously carried out to remove components with an undesired odor.

It is advantageous, before the stripping with steam or before the steam distillation, to neutralize the polymerization solution with one of the neutralizing agents specified below to at least 20%, in particular to at least 25% (based on the total amount of neutralizable groups).

In a further embodiment of the invention, the concentrated polymer/solvent mixture obtained after the steam distillation is diluted with alcohol, in particular ethanol.

The monomers used for the polymerization are preferably converted to at least 95%, particularly preferably to at least 99% and in particular to at least 99.9% (degree of polymerization).

The polymers present in solution after the polymerization can be converted into powders by customary drying methods known to the person skilled in the art. Preferred methods are spray-drying, spray fluidized-bed drying, drum-drying and belt-drying. Freeze-drying and freeze-concentration can likewise be used.

If desired, solvents can also be partially or completely removed by customary methods, e.g. distillation at reduced pressure.

The polymers produced by the method according to the invention may be anionic or anionogenic polymers. For use in cosmetic preparations; it is advantageous if the acid groups of these polymers are partially or completely neutralized with a base since the salts of the polymers obtained generally have better solubility or dispersibility in water than do the unneutralized polymers.

Bases for the neutralization of these polymers which may be used are alkali metal bases, such as sodium hydroxide solution, potassium hydroxide solution, sodium carbonate, sodium hydrogencarbonate, potassium carbonate or potassium hydrogencarbonate, and alkaline earth metal bases, such as calcium hydroxide, calcium oxide, magnesium hydroxide or magnesium carbonate, and ammonia or amines.

Suitable amines are, for example, C₁-C₆-alkylamines, preferably n-propylamine and n-butylamine, dialkylamines, preferably diethylpropylamine and dipropylmethylamine, trialkylamines, preferably triethylamine and triisopropylamine, C₁-C₆-alkyldiethanolamines, preferably methyl- or ethyldiethanolamine and di-C₁-C₆-alkylethanolamines. Particularly for use in cosmetic preparations, in particular in hair-treatment compositions, 2-amino-2-methyl-1-propanol (AMP), 2-amino-2-ethylpropane-1,3-diol, diethylaminopropylamine, triisopropanolamine and triethanrolamine have proven useful for neutralizing polymers comprising acid groups. If the neutralizing agent used is triethanolamine, the viscosity of the mixture present after the steam distillation and before the renewed addition of alcohol is lower than, for example, if AMP is used.

The neutralization of the polymers comprising acid groups can also be carried out with the help of mixtures of two or more bases, e.g. mixtures of sodium hydroxide solution or potassium hydroxide solution and triisopropanolamine.

Further suitable neutralizing agents are disclosed in WO 03/099253, p. 2 I. 1 to p. 3, I. 6, to the entire contents of which reference is hereby made.

Preferably, the polymers are neutralized when polymerization is complete, i.e. at a monomer conversion of at least 99% by weight.

The degree of neutralization can be 5 to 1.00%, preferably 30 to 95%, depending on the intended use. However, the neutralizing agent can also be added in more than an equivalent amount.

### Cosmetic Preparations

Stricter environmental regulations and a growing ecological awareness increasingly demand ever lower fractions of volatile organic components (VOCs) in cosmetic aerosol preparations such as, for example, aerosol hair sprays.

The VOC content in hair sprays is essentially determined by the nonaqueous solvents and the propellants. For this reason, instead of nonaqueous solvents, recourse is currently and increasingly being made to water as solvent. However, this replacement of the organic solvents entails a number of problems. Thus, formulations of the film-forming polymers known from the prior art which satisfy the corresponding VOC regulations are not, for example, sprayable, or are only sprayable following further dilution and are thus only of limited suitability for use in hair sprays. Polymer films which are formed from such preparations sometimes do not have the required mechanical quality and thus have inadequate setting effect and poor hold for the hair. The copolymers produced by the method according to the invention are exceptionally suitable for producing cosmetic, in particular skin cosmetic and/or hair cosmetic, preparations. They serve here, for example, as polymeric film formers. They can be used and formulated universally in a very wide variety of cosmetic, preferably hair cosmetic, preparations and are compatible with customary components.

The copolymers are advantageously suitable for producing elastic hairstyles coupled with strong hold, even at high atmospheric humidity. The copolymers are notable for good propellant gas compatibility, good solubility in aqueous/alcoholic solvent mixtures, in particular for the suitability for use as optically clear low-VOC formulations and for good ability to be washed out and ability to be combed out without flaking effect. In addition, they improve hair treated therewith in its sensorially perceptible properties, such as feel, volume or handleability. Hair spray formulations based on the copolymers produced by the method according to the invention are notable for good sprayability and good rheological properties and extremely low stickiness of the resulting films. The cosmetic, preferably hair cosmetic, preparations comprising the copolymers do not have a tendency for foaming following application. Besides the good compatibility with the customary cosmetic ingredients, the applied copolymer films-dry rapidly.

The present invention accordingly further provides the use of the copolymers obtainable by the method according to the invention in cosmetic preparations, and also such cosmetic preparations per se.

### Cosmetically Acceptable Carrier B

The cosmetic preparations are preferably aqueous, preparations which comprise at least 10% by weight, preferably at least 20% by weight and particularly preferably at least 30% by weight, of water. Preferably, the cosmetic preparations according to the invention comprise at most 80% by weight (VOC-80), preferably at most 55% by weight (VOC-55) of volatile-organic constituents.

The invention accordingly provides cosmetic preparations in which the fraction of volatile organic components is at most 55% by weight, based on the cosmetic preparation.

Besides water and the copolymers obtainable by the method according to the invention, the cosmetic preparations further have, at least one cosmetically acceptable carrier B which is chosen from
i) water-miscible organic solvents, preferably C₂-C₄-alkanols, in particular ethanol,
ii) oils, fats, waxes,
iii) esters of C₆-C₃₀-monocarboxylic acids with mono-, di- or trihydric alcohols different from ii),
iv) saturated acyclic and cyclic hydrocarbons,
v) fatty acids,
vi) fatty alcohols,
vii) propellants (propellant gases) and
viii) mixtures thereof.

Suitable carriers B and further active ingredients and additives to be used advantageously are described in detail below.

Suitable cosmetically and pharmaceutically compatible oil and fat components are described in Karl-Heinz Schrader, Grundlagen und Rezepturen der Kosmetika [Fundamentals and Formulations of Cosmetics], 2nd edition, Veriag Hüthig, Heidelberg, pp. 319-355, to which reference is hereby made.

As cosmetically acceptable carrier B, the cosmetic preparations can, for example, have an oil or fat component which is chosen from: hydrocarbons of low polarity, such as mineral oils; linear saturated hydrocarbons, preferably having more than 8 carbon atoms, such as tetradecane, hexadecane, octadecane etc.; cyclic hydrocarbons, such as decahydronaphthalene; branched hydrocarbons; animal and vegetable oils; waxes; wax esters; vaseline; esters, preferably esters of fatty acids, such as, for example, the esters of C₁-C₂₄-monoalcohols with C₁-C₂₂-monocarboxylic acids, such as isopropyl isostearate, n-propyl myristate, isopropyl myristate, n-propyl palmitate, isopropyl palmitate, hexacosanyl palmitate, octacosanyl palmitate, triacontanyl palmitate, dotriacontanyl palmitate, tetratriacontanyl palmitate, hexacosanyl stearate, octacosanyl stearate, triacontanyl stearate, dotriacontanyl stearate, tetratriacontanyl stearate; salicylates, such as C₁-C₁₀-salicylates, e.g. octyl salicylate; benzoate esters, such as C₁₀-C₁₅-alkyl benzoates, benzyl benzoate; other cosmetic esters, such as fatty acid triglycerides, propylene glycol monolaurate, polyethylene glycol monolaurate, C₁₀-C₁₅-alkyl lactates, etc. and mixtures thereof.

Suitable silicone oils B) are, for example, linear polydimethylsiloxanes, poly(methylphenylsiloxanes), cyclic siloxanes and mixtures thereof. The number-average molecular weight of the polydimethylsiloxanes and poly(methylphenylsiloxanes) is preferably in a range from about 1000 to 150 000 g/mol. Preferred cyclic siloxanes have 4- to 8-membered rings. Suitable cyclic siloxanes are commercially available, for example, under the name cyclomethicone.

Preferred oil or fat components B) are chosen from paraffin and paraffin oils; vaseline; natural fats and oils, such as castor oil, soya oil, peanut oil, olive oil, sunflower oil, sesame oil, avocado oil, cocoa butter, almond oil, peach kernel oil, ricinus oil, cod-liver oil, pig fat, spermaceti, spermaceti oil, sperm oil, wheat germ oil, macademia nut oil, evening primrose oil, jojoba oil; fatty alcohols, such as lauryl-alcohol, myristyl alcohol, cetyl alcohol, stearyl alcohol, oleyl alcohol, cetyl alcohol; fatty acids, such as myristic acid, stearic acid, palmitic acid, oleic-acid, linoleic acid, linolenic acid and saturated, unsaturated and substituted fatty acids different therefrom; waxes, such as beeswax, carnauba wax, candelilla wax, spermaceti, and mixtures of the abovementioned oil and fat components.

Suitable hydrophilic carriers B) are chosen from water, 1-, 2- or polyhydric alcohols having preferably 1 to 8 carbon atoms, such as ethanol, n-propanol, isopropanol, propylene glycol, glycerol, sorbitol, etc.

The cosmetic preparations may be skin cosmetic, hair cosmetic or dermatological, hygiene or pharmaceutical preparations. On account of their film-forming and flexible properties, the copolymers which can be produced by the method according to the invention are particularly suitable as additives for hair cosmetics and skin cosmetics.

Preferably, the cosmetic preparations which comprise the copolymers according to the invention are in the form of spray, gel, foam, ointment, cream, emulsion, suspension, lotion, milk or paste. If desired, liposomes or microspheres can also be used.

Preferably, the cosmetic compositions according to the invention comprise at least one copolymer according to the invention, at least one carrier B as defined above and at least one constituent different therefrom which is preferably chosen from cosmetically active ingredients, emulsifiers, surfactants, preservatives, perfume oils, thickeners, hair polymers, hair and skin conditioners, graft polymers, water-soluble or dispersible silicone-containing polymers, photoprotective agents, bleaches, gel formers, care agents, colorants, tinting agents, tanning agents, dyes, pigments, consistency regulators, humectants, refatting agents, collagen, protein hydrolyzates, lipids, antioxidants, antifoams, antistats, emollients and softeners.

The preparations according to the invention preferably have a pH of from 2.0 to 9.3.

The pH range is particularly preferably between 4 and 8. Additional cosolvents which may be present are organic-solvents or a mixture of solvents with a boiling point below 400° C. in an amount of from 0.1 to 15% by weight, preferably from 1 to 10% by weight. Particularly suitable additional cosolvents are unbranched or branched hydrocarbons, such as pentane, hexane, isopentane and cyclic hydrocarbons, such as cyclo-pentane and cyclohexane. Further particularly preferred water-soluble solvents are glycerol, ethylene glycol and propylene glycol in an amount up to 30% by weight.

In one preferred embodiment of the invention, the cosmetic preparations have a fraction of volatile organic components of at most 80% by weight, preferably at most 55% by weight and in particular at most 35% by weight. A preferred subject matter is thus cosmetic, preferably hair cosmetic, preparations which correspond to the low-VOC standard, i.e. VOC-80 or VOC-55 standard.

Preference is given to the use of the copolymers, in particular in hair spray preparations, which comprise the following constituents:
partially or completely neutralized copolymer according to the invention;
water;
cosmetically customary organic solvent, such as, for example, ethanol, isopropanol and dimethoxymethane, in addition also acetone, n-propanol, n-butanol, 2-methoxypropan-1-ol, n-pentane, n-hexane, cyclohexane, n-heptane, n-octane or dichloromethane or mixtures thereof;
cosmetically customary propellant, such as, for example, n-propane, isopropane, n-butane, isobutane, 2,2-dimethylbutane, n-pentane, isopentane, dimethyl ether, difluoroethane, fluorotrichloromethane, dichlorodifluoromethane or dichlorotetrafluoroethane, HFC-152 A (1,1-difluoroethane), HFC-134a (1,1,2,2-tetrafluoroethane), N₂, N₂O and CO or mixtures thereof.

For neutralizing the copolymers obtainable by the method according to the invention and for adjusting the pH of the cosmetic, preferably hair cosmetic, preparations, the agents already specified above are suitable.

Alkanolanines are advantageously used. Examples are 2-amino-2-methyl-1-propanol (AMP), diethanolamine, diisopropanolamine, ethanolamine, methylethanolamine, N-lauryldiethanolamine, triethanolamine, triisopropanolamine, and also those specified in WO 03/099253, p. 2 I. 1 to p. 3, I. 6. Accordingly, it is possible to use alkanolamines carrying either primary amino groups or secondary amino groups.

Furthermore, alkali metal hydroxides (e.g. NaOH, preferably KOH) and other bases can be used for the neutralization (e.g. histidine, arginine, lysine, ethylenediamine, diethylenetriamine, melamine, benzoguanamine). All of the stated bases can be used on their own or as a mixture with other bases for the neutralization of acid-containing cosmetic products.

In one preferred embodiment of the invention, amines comprising hydroxy groups from the group consisting of N,N-dimethylethanolamine, N-methyldiethanolamine, triethanolamine, 2-amino-2-methylpropanol and mixtures thereof, and those specified in WO 03/099253, p. 2 I. 1 to p. 3, I. 6 are chosen for the neutralization.

The present invention accordingly provides aqueous cosmetic, preferably skin cosmetic and/or hair cosmetic, preparations which, besides the at least one copolymer obtainable by the method according to the invention and the carrier B, comprise at least also one active ingredient or additive chosen from the group consisting of viscosity-modifying substances, hair care substances, hair-setting substances, silicone compounds, photoprotective substances, fats, oils, waxes, preservatives, pigments, soluble dyes, particulate substances and surfactants.

In one preferred embodiment, such hair cosmetic formulations comprise

i) 0.05 to 20% by weight of at least one copolymer as described above,
ii) 20 to 99.95% by weight of water and/or alcohol,
iii) 0 to 50% by weight of at least one propellant gas,
iv) 0 to 5% by weight of at least one emulsifier,
v) 0 to 3% by weight of at least one thickener, and
vi) up to 25% by weight of further constituents.

Alcohol is understood as meaning all of the abovementioned alcohols customary in cosmetics, preferably ethanol, isopropanol, n-propanol, where ethanol is particularly preferred.

### Propellants (Propellant Gases)

Of the specified compounds, the propellants (propellant gases) used are primarily the hydrocarbons, in particular propane, n-butane, n-pentane and mixtures thereof, and also dimethyl ether and difluoroethane. If appropriate, one or more of the specified chlorinated hydrocarbons are co-used in propellant mixtures, but only in small amounts, for example up to 20% by weight, based on the propellant mixture.

The cosmetic preparations are also particularly suitable for pump spray preparations without the addition of propellants or else for aerosol sprays with customary compressed gases such as nitrogen, compressed air or carbon dioxide as propellant. A water-containing standard aerosol spray formulation comprises, for example, the following constituents:
copolymer neutralized to 100%
alcohol
water
dimethyl ether and/or propane/n-butane and/or propane/isobutane,

Here, the total amount of volatile organic components is preferably at most 80% by weight, particularly preferably at most 55% by weight, of the preparation.

Preferably, the cosmetic preparations comprise at least one copolymer as described above; at least one cosmetically acceptable carrier B as defined above and at least one further active ingredient or additive different therefrom which is chosen from cosmetically active ingredients, emulsifiers, surfactants, preservatives, perfume oils, thickeners, hair polymers, hair conditioners, graft polymers, water-soluble or dispersible silicone-containing polymers, photoprotective agents, bleaches, gel formers, care agents, colorants, tinting agents, tanning agents, dyes, pigments, consistency regulators, humectants, refatting agents, collagen, protein hydrolyzates, lipids, antioxidants, antifoams, antistats, emollients, lanolin components, protein hydrolyzates and softeners.

### Further Polymers

In order to adjust the properties of cosmetic preparations in a targeted way, it may be advantageous to use the copolymers obtainable by the method according to the invention in a mixture with further (hair) cosmetically customary polymers.

In a further preferred embodiment, the cosmetic preparation comprises 0.01 to 15% by weight, preferably 0.5 to 10% by weight, of at least one further synthetic or natural nonionic, preferably a film-forming polymer. Natural polymers are also understood as meaning chemically modified polymers of natural origin. Film-forming polymers are understood as meaning those polymers which, when applied in 0.01 to 5% strength aqueous, alcoholic or aqueous-alcoholic solution, are able to deposit a polymer film on the hair.

Suitable as such further customary polymers for this purpose are, for example, anionic, cationic, amphoteric, zwitterionic and neutral polymers. Such polymers are known to the person skilled in the art and require no further explanation.

### Measurement Methods

### Determination of the K Value

The K values are measured in accordance with Fikentscher, Cellulosechemie, vol. 13, pp. 58 to 64 (1932) at 25° C. in N-methylpyrrolidone (NMP) solution and are a measure of the molecular weight. The NMP solution of the polymers comprises 1 g of polymer in 100 ml of solution.

If the polymers are in the form of aqueous-dispersions, corresponding amounts of the dispersion are topped; up to 100 ml with NMP depending on the polymer content of the dispersion, giving concentrations of 1 g of polymer in 100 ml of solution.

The K value is measured in a micro-Ubbelohde capillary type M Ic from Schott.

Determination of the droplet size distribution (DSD) Means of Malvern® Scattered Light Analysis

The droplet size distribution was determined using particle size measurement system for detecting liquid aerosols “Malvern® Master Sizer X” (Malvern Instruments Inc., Southborough Mass., USA).

### Measurement Principle:

The measurement system is based on the method of laser light diffraction at the particle, which is suitable not only for spray analysis (aerosols, pump sprays), but also for determining the size of solids, suspensions and emulsions in the size range from 0.1 μm to 2000 μm.

A particle collective (=droplet) is illuminated by a laser. At each droplet, some of the incident laser light is scattered. This light is captured on a multi-element detector and the corresponding light energy distribution is determined. This data is used to calculate the corresponding particle distribution via the evaluation, software.

### Procedure:

The aerosols were sprayed at a distance of 29.5 cm from the laser beam. The spray cone was at right angles to the laser beam. Spray device: spray head Kosmos 0.020D vortex 0.018″ 21 6443-20, valve: DPV 33876 (Precision Valve)

Before each measurement, the aerosol cans were attached to a firmly installed holding device, thus meaning that all of the aerosols to be tested were measured at exactly the same distance.

Before the actual particle measurement, a so-called “background measurement” was carried out. By doing so, the effects of dust and other contaminants within the measurement range were eliminated.

The aerosol was then sprayed into the test space. The entire particle volume was detected for a test period of 2 s and evaluated.

### Evaluation:

Evaluation is made via a tabular depiction over 32 class widths from 0.5 μm to 2000 μm and a graphical depiction of the particle size distribution.

Since the spray experiments are approximately uniform distributions, the mean diameter D (v,0.5) is given. This numerical value indicates that 50% of the total particle volume measured is below this value.

For readily sprayable aerosol systems in the cosmetics sector, this value is in the range from 30 μm to 80 μm, depending on the polymer content, geometry of the valve and spray head, solvent ratio and amounts of propellant gas.

### Determination of the Setting (Flexural Rigidity)

The setting of polymeric film formers was measured not only by a subjective assessment (so-called “hand test”), but also physically by measuring the flexural rigidity of thin sections of hair (in each case about 3 g and 24 cm in length). For this, the weighed, dry hair sections were dipped into a 3.0% strength by weight solution of the polymer in question (dissolved in ethanol/water 55:45 w/w), with triple immersion and removal and subsequent squeezing between filter paper ensuring uniform wetting of the sections of hair and distribution of the polymer solution. The excess polymer solution was then squeezed out between thumb and forefinger and the sections of hair were shaped by hand so that they had an approximately round cross section. They were stored overnight at 20° C. and 65% relative humidity in a climatically controlled room. The tests were carried out in the climatically-controlled room at 20° C. and 65% relative humidity using a stress/strain testing device. The section of hair was placed symmetrically at the ends of two cylindrical rolls of the sample holder. In exactly the middle, the section was then bent from above using a rounded punch until the polymer film broke (ca. 40 mm). The force required for this (Fₘₐₓ) was determined using a weighing cell (50 N). Here, one measurement value represents the arithmetic mean from the individual measurements on 5 to 10 identically treated hair sections. The values ascertained in this way were compared to those for a standard commercial comparison polymer (as described) and given in %.

### Determination of the Ability to be Washed Out

A section of hair treated with polymer analogously to the determination of the setting was washed in a ca. 37° C.-hot Texapon®NSO solution (6 ml of Texapon®NSO (28% strength) in 1 l of warm water) for ca. 15 seconds by dipping it in and squeezing it 5 times. The section of hair was then rinsed until clear and treated again in the same way. The section of hair was then squeezed thoroughly on filter paper and left to dry overnight. The dry section of hair was put in rollers and analyzed for residues.

### Determination of the Curl Retention

### Basic Formulation: (Aerosol Hair Spray)

5% by wt. of active ingredient of polymer to be tested (100% neutr. with AMP)

15% by wt. of ethanol

40% by wt. of water

40% by wt. of dimethyl ether.

To determine the curl retention, hair sections ca. 2 g in weight and 15.5 cm in length and comprising mid-brown, Caucasian human hair were used.

### Treatment of the Sections of Hair:

The hair sections were washed twice with an aqueous Texapon®NSO solution. The sections of hair were then rinsed with warm water until no more foaming was evident and after-rinsed with demineralized water, combed and laid to dry on filter paper.

To produce a water wave, the sections of hair are placed into a solution of ethanol and water (1:1) to swell for 15 minutes.

The section of hair is carefully combed prior to curl preparation. The section of hair is attached to a Plexiglass rod using a rubber band. It is then combed and wound in a spiral. Using a cotton cloth and rubber band, the curl is firmly fixed and dried overnight at 70° C. The cooled curl retention sections are carefully opened and slipped off the Plexiglass rod without deforming the water wave. From a distance of 15 cm, 1.8 g of the aerosol hair spray produced according to the composition above are sprayed uniformly onto the curl. The curl is rotated evenly during this. The curls are dried in the horizontal position for 1 h at room temperature. After drying, the curls are secured in a clamp. Using a ruler, at the start the starting length of the curls is read off and the length extension during humid storing is monitored. After storage for 5; h at 25° C. and 90% relative humidity in the climatically controlled chamber, the length of the curl achieved is again read off and the curl retention is calculated according to the following equation:

$$Curl Retention in \% = \frac{L - {L}_{t}}{L - {L}_{o}} * 100$$

L=length of hair (15.5 cm)
L₀=length of the hair curl after drying
Lₜ=length of the hair curl after climatic treatment

The mean value from the 5 individual measurements is given as curl retention.

### Determination of the Stickiness

Firstly, a clear, 20% strength by weight ethanolic or ethanolic/aqueous solution of the polymer to be characterized is prepared. In order to obtain a clear solution, it is necessary, if appropriate, to neutralize the polymer. A doctor knife (120 μm split width) is then used to apply a film of the polymer from the ethanolic or ethanolic/aqueous solution on a glass plate. This rectangular glass plate had a length of ca. 20 cm and a width of ca. 6.5 m. The polymer film applied thereto had a length of about 16 to 18 cm and a width of ca 5.5 cm.

The film is then dried in the air for ca. 10 hours and then stored in a climatically controlled cabinet for a further 12 hours at 20° C. and 80% relative humidity.

Then, under these conditions, in the climatically controlled cabinet, a plastic carbon ribbon (e.g. Pelikan®2060, 50 mm wide) located on a round rubber punch (diameter 400 mm, Shore A hardness 60±5) is pressed into the polymer film with a force of ca. 250 N for 10 seconds.

The amount of black pigment which remains adhering to the polymer film after the punch has been removed corresponds to the stickiness of the film. A visual assessment of the black coloration of the film is made. The assessment scale ranges from 0 to 5, where 0 is not sticky and 5 is very considerably sticky.

### Determination of the Appearance of the Aerosol Formulation

The preparation of 5% by weight of the particular polymer neutralized with AMP, 40% by weight of DME, 150% by weight of ethanol and 40% by weight of water were poured into a transparent glass aerosol container. The clarity of the resulting liquid/propellant gas mixture was then assessed visually.

### EXAMPLES

### Abbreviations

MMA methyl methacrylate
MM methacrylic acid
AA acrylic acid
Preparation of Urethane Acrylate 1 (Component iii)

In a round-bottomed flask, 672.0 g of a polyester of adipic acid and neopentyl glycol with an OH number of about 200, 140.0 g of hydroxyethyl acrylate, 0.6 g of hydroquinone monomethyl ether, 1.20 g of 2,6-di-tert-butyl-4-methylphenol, 0.12 g of tetrabutyl orthotitanate were initially introduced and heated to 50° C. 400.0 g of isophorone diisocyanate were then added dropwise over the course of 30 minutes. The mixture was left to react for a further 7 hours at 90-95° C., during which the NCO content dropped to 0.56%. The mixture was cooled to 60° C., then 520.0 g of ethanol were added and the mixture was left to react further for about 2 hours at 65-70° C. until the isocyanate content (NCO value) had dropped to 0. The resin obtained was filtered over a 50 μm filter and bottled.

Preparation of Urethane-Acrylate 2 (Component iii)

In a round-bottomed flask, 672.0 g of a polyester of adipic acid and neopentyl glycol with an OH number of about 200, 140.0 g of hydroxyethyl acrylate, 0.6 g of hydroquinone monomethyl ether, 1.21 g of 2,6-di-tert-butyl-4-methylphenol, 0.12 g of cesium acetate were initially introduced and heated to 50° C., 400.0 g of isophorone diisocyanate were then added dropwise over the course of 30 minutes. The mixture was left to react for a further 7 hours at 90-95° C. during which the NCO content dropped to 0.56%. It was cooled to 60° C., then 520.0 g of ethanol were added and the mixture was left to react further for about 2 hours at 65-70° C. until the isocyanate content (NCO value) had dropped to 0. The resin obtained was filtered over a 50 μm filter and bottled.

Preparation of Urethane Acrylate 3 (Component iii)

In a round-bottomed flask, 672.0 g of a polyester of adipic acid and neopentyl glycol with an OH number of about 200, 140.0 g of hydroxyethyl acrylate, 0.6 g of hydroquinone monomethyl ether, 1.21 g of 2,6-di-tert-butyl-4-methylphenol were initially introduced and heated to 50° C. 400.0 g of isophorone diisocyanate were then added dropwise over the course of 30 minutes. The mixture was left to react for a further 20 hours at 90-95° C., during which the NCO content dropped to 0.1%. It was cooled to 60° C., then 10.0 g of methanol were added and the mixture was left to react further for 4 hours at 90-95° C. until the isocyanate content (NCO value) had dropped to 0. The resin obtained was mixed at room temperature with 510.0 g of tripropylene glycol diacrylate and filtered over a 50 μm filter and bottled.

Urethane Acrylate 5 (Component iii)

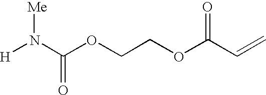

Urethane Acrylate 6 (Component iii)

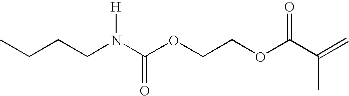

Urethane Acrylate 7 (Component iii)

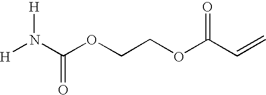

### Polymer A:

Feed 1

| | |
|---|---|
| 234.00 g | methyl methacrylate (MMA) |
| 30.00 g | methacrylic acid (MAA) |
| 30.00 g | acrylic acid (AA) |
| 6.00 g | urethane acrylate 3 (UA 3) |

Feed 2

| | |
|---|---|
| 6.00 g | Wako ® V-50 (2,2′-azobis(2-methylpropionamide) |
| | dihydrochloride) |
| 270.00 g | ethanol |
| 204.00 g | dem. water (dem. = completely demineralized) |

As the initial charge, 15.00 g of feed 1 and 24.00 g of feed 2 were mixed with 120.0 g of cosmetic ethanol and 90.00 g of dem. water in a 1 l glass reactor. This initial charge was heated to reflux under a nitrogen atmosphere. After the reflux temperature had been reached, feeds 1 and 2 were started together. Feed 1 was metered in over 3 hours and feed 2 was metered in over 4 hours under reflux. The reaction mixture was further polymerized under reflux for 2 hours. Then, feed 3 (1.05 g of sodium peroxodisulfate, 12.00 g of ethanol cosm., 8.00 g of dem. water) was metered in over 30 minutes and the mixture was after-polymerized under reflux for 2 hours. Feed 4 (1.05 g of sodium peroxodisulfate, 12.00 g of ethanol cosm., 8.00 g of dem. water) was then metered in over 30 minutes and the mixture was after-polymerized under reflux again for 2 hours.

### Polymer B:

Feed 1

| | |
|---|---|
| 117.00 g | methyl methacrylate |
| 15.00 g | methacrylic acid |
| 15.00 g | acrylic acid |
| 3.00 g | Laromer ® UA 19 T |

Feed 2

| | |
|---|---|
| 3.00 g | sodium peroxodisulfate |
| 135.00 g | ethanol cosm. |
| 102.00 g | dem. water |

As the initial charge, 2.34 g of feed 1 and 24.00 g of feed 2 were mixed with 60.0 g of cosmetic ethanol and 45.00 g of dem. water in a 1 l glass reactor. This initial charge was heated to reflux under a nitrogen atmosphere. After the reflux temperature had been reached, feeds 1 and 2 were started together. Feed 1 was metered in under reflux over 3 hours and feed 2 was metered in under reflux over 4 hours. The reaction mixture was further polymerized under reflux for 2 hours. Feed 3 (0.70 g of sodium peroxodisulfate, 6.00 g of ethanol cosm., 4.00 g of dem. water) was metered in over 30 minutes and after-polymerized under reflux for 2 hours. Feed 4 (0.70 g of sodium peroxodisulfate, 6.00 g of ethanol cosm., 4.00 g of dem. water) was then metered in over 30 minutes and after-polymerized under reflux again for 2 hours.

### Polymer C

With stirring at 20° C., the following feeds were prepared.

Feed 1

| | |
|---|---|
| 120 g | MMA |
| 15 g | MAA |
| 15 g | AA |

Feed 2

| | |
|---|---|
| 3 g | sodium peroxodisulfate |
| 135 g | ethanol |
| 102 g | wasser |

At 20° C., a mixture of 100 g of ethanol, 15% by weight of the total amount of feed 1 and 15% by weight of the total amount of feed 2 was prepared. The mixture was heated to 78° C. under atmospheric pressure. While maintaining the polymerization temperature, after 78° C. had been reached, feed 1 and feed 2 were started. Feed 1 was metered in over the course of 3 h, and feed 2 was, metered in over the course of 4 h with a constant feed stream. When feed 2 was complete, the reaction mixture was kept at 78° C. for a further 2 hours and then cooled to room temperature (about 20° C.).

### Polymer D

The polymer was prepared analogously to polymer A. Here, feed 3 (0.60 g of sodium peroxodisulfate, 12.00 g of ethanol cosm., 8.00 g of dem. water) was metered in over 30 minutes and after-polymerized under reflux for 2 hours. Feed 4 (0.60 g of sodium peroxodisulfate, 12.00 g of ethanol cosm., 8.00 g of dem. water) was then metered in over 30 minutes and the mixture was after-polymerized under reflux again for 2 hours

### Polymer E

Feed 1

| | |
|---|---|
| 228.15 g | methyl methacrylate |
| 29.25 g | methacrylic acid |
| 29.25 g | acrylic acid |
| 5.85 g | urethane acrylate 1 |

Feed 2

| | |
|---|---|
| 4.70 g | sodium peroxodisulfate |
| 199.0 g | dem. water |

As the initial charge, 14.60 g of feed 1 and 10.20 g of feed 2 were mixed with 403.00 g of isopropanol and 87.80 g of dem. water in a 1 l glass reactor. This initial charge was heated to 80° C. under a nitrogen atmosphere. After the temperature had been reached, feeds 1 and 2 were started together. Feed 1 was metered in over 3 hours and feed 2 was metered in over 4 hours. The reaction mixture was further polymerized under reflux for 2 hours. Feed 3 (0.88 g of sodium peroxodisulfate, 6.00 g of dem. water) was then metered in and the mixture was after-polymerized for 2=hours. Feed 4 (0.88 g of sodium peroxodisulfate, 6.00 g of dem. water) was then metered in and the mixture was after-polymerized again for 2 hours.

### Polymer F

Feed 1

| | |
|---|---|
| 234.0 g | methyl methacrylate |
| 30.0 g | methacrylic acid |
| 30.0 g | acrylic acid |
| 6.0 g | urethane acrylate 1 |

Feed 2

| | |
|---|---|
| 3.60 g | Wako ® V-50 (2,2′-azobis(2-methylpropionamide) |
| | dihydrochloride) |
| 270.0 g | isopropanol |
| 204.0 g | dem. water |

As the initial charge, 15.0 g of feed 1 and 23.90 g, of feed 2 were mixed with 120.0 g of isopropanol and 90.0 g of dem. water in a 1 l glass reactor. This initial charge was heated to 80° C. under a nitrogen atmosphere. After the temperature had been reached, feeds 1 and 2 were started together. Feed 1 was metered in over the course of 3 hours and feed 2 was metered in over the course of 4 hours. The reaction mixture was further polymerized under reflux for 2 hours. Feed 3 (0.60 g of sodium peroxodisulfate, 8.00 g of dem. water, 12.0 g of isopropanol) was then metered in over the course of 30 min and the mixture was after-polymerized for 2 hours. Feed 4 (0.60 g of sodium peroxodisulfate, 8.00 g of dem. water, 12.0 g of isopropanol) was then metered in over the course of 30 min and the mixture was after-polymerized for 4 hours.

The solution obtained had a solids content of 32.6% (residual monomers: table). This solution was partially neutralized with 27.90 g of 2-amino-2-methyl-1-propanol. 350.0 g of this solution were subjected to steam distillation (15 min). This gave 228.0 g of a white concentrated mixture which was then diluted with 70.0 g of ethanol.

### Polymer G

Feed 1

| | |
|---|---|
| 234.0 g | methyl methacrylate |
| 30.0 g | methacrylic acid |
| 30.0 g | acrylic acid |
| 6.0 g | urethane acrylate 1 |

Feed 2

| | |
|---|---|
| 3.60 g | Wako ® V-50 (2,2′-azobis(2-methylpropionamide) |
| | dihydrochloride) |
| 204.0 g | dem. water |

As the initial charge, 15.0 g of feed 1 and 10.40 g of feed 2 were mixed with 414.0 g of isopropanol and 90.0 g of dem. water in a 2 l glass reactor. This initial charge was heated to 80° C. under a nitrogen atmosphere. After the temperature had been reached, feeds 1 and 2 were started together. Feed 1 was metered in over the course of 3 hours and feed 2 was metered in over the course of 4 hours. The reaction mixture was further polymerized under reflux for 2 hours. Feed 3 (0.90 g of sodium peroxodisulfate, 8.00 g of dem. water) was then metered in over the course of 30 min and the mixture was after-polymerized for 2 hours. Feed 4 (0.90 g of sodium peroxodisulfate, 8.00 g of dem. water) was then metered in over the course of 30 min and the mixture was after-polymerized again for 2 hours.

The solution obtained had a solids content of 30.9% by weight. 345.0 g of this solution were partially neutralized with 12.0 g of 2-amino-2-methyl-1-propanol and, subjected to steam distillation (15 min). The concentrated mixture was then diluted with 106.0 g of ethanol to give a solution with a solids content of 27.0% by weight.

### Polymer H

| | | |
|---|---|---|
| Feed 1 | 210.0 g | methyl methacrylate |
| | 36.0 g | methacrylic acid |
| | 36.0 g | acrylic acid |
| | 37.5 g | ethyl dimethyl (2-((2-methyl-1-oxoallyl)- |
| | | oxy)ethyl)ammonium ethyl sulphate (48% aqueous |
| | | solution) - acrylate 4 |
| Feed 2 | 3.60 g | Wako ® V-50 |
| | 270.0 g | isopropanol |
| | 184.5 g | dem. water |

As the initial charge, 16.0 g of feed 1 and 23.0 g of feed 2 were mixed with 120.0 g of isopropanol and 90.0 g of dem. water in a 2 l glass reactor. This initial charge was heated to 80° C. under a nitrogen atmosphere. After the temperature had been reached, feeds 1 and 2 were started together. Feed 1 was metered in over the course of 3 hours and feed 2 was metered in over the course of 4 hours. The reaction mixture was further polymerized under reflux for 2 hours. Feed 3 (0.60 g of sodium peroxodisulfate, 8.00 g of dem. water, 12.0 g of isopropanol) was then metered in over the course of 30 min and the mixture was after-polymerized for 2 hours. Feed 4 (0.60 g of sodium peroxodisulfate, 8.00 g of dem. water, 12.0 g of isopropanol) was then metered in over the course of 30 min and the mixture was after-polymerized again for 4 hours.

The solution obtained has a solids content of 32.6% (residual monomers: table). 803.0 g of this solution were partially neutralized with 35.50 g of 2-amino-2-methyl-1-propanol and subjected to steam distillation (15 min). This gave a white viscous mixture which was diluted with ethanol cosm. to give a solution with a solids content of 36.4% by weight.

### Polymer I

Feed 1

| | |
|---|---|
| 700.0 g | methyl methacrylate |
| 90.0 g | methacrylic acid |
| 90.0 g | acrylic acid |
| 18.0 g | urethane acrylate 1 |
| 360.0 g | isopropanol |

Feed 2

| | |
|---|---|
| 10.70 g | Wako ® V 50 (2,2′-azobis(2-methylpropionamide) |
| | dihydrochloride) |
| 82.0 g | dem. water |
| 32.0 g | isopropanol |

As the initial charge, 63.00 g of feed 1 were mixed with 803.0 g of isopropanol and 746.0 g of dem. water in a 5 l stainless steel reactor. This initial charge was purged 3 times with a nitrogen atmosphere (5.0 bar) and then heated to 80° C. at 0.5 bar. 6.30 μg of feed 2 were then added. After 10 minutes, feeds 1 and 2 were started together. Feed 1 was metered in over the course of 3 hours and feed 2 was metered in over the course of 4 hours at 80° C. under autogenous pressure. The reaction mixture was further polymerized for 2 hours at 80° C. under autogenous pressure. The temperature was then increased to 90° C. under autogenous pressure and feed 3 (2.70 g of sodium peroxodisulfate, 36.00 g of dem. water) was metered in over the course of 30 minutes and the mixture was after-polymerized at 90° C. for 2 hours, under autogenous pressure. Feed 4 (2.70 g of sodium peroxodisulfate, 36.00 g of dem. water) was then metered in over the course of 30 minutes and the mixture was after-polymerized at 90° C. again for 2 hours under autogenous pressure.

The solution obtained (solids content of 32.0% by weight) was partially neutralized with 131.10 g of 2-amino-2-methyl-1-propanol and subjected to steam-distillation. The mixture was then diluted with ethanol cosm. to give a solution with a solids-content of 35.7% by weight.

### Polymer J

The polymer was synthesized analogously to polymer I.

968.0 g of the solution were partially neutralized with 71.10 g of triethanolamine and subjected to steam distillation (15, min). The viscous solution was then diluted with ethanol cosm. to give a solution with a solids content of 32.5% by weight.

### Polymer K

| | | |
|---|---|---|
| Feed 1 | 228.0 g | methyl methacrylate |
| | 30.0 g | methacrylic acid |
| | 30.0 g | acrylic acid |
| | 12.0 g | Ebecryl ® CL 1039 |
| Feed 2 | 6.00 g | Wako ® V-50 |
| | 200.0 g | dem. water |
| | 270.0 g | ethanol cosm. |

As the initial charge, 15.0 g of feed 1 and 24.0 g of feed 2 were mixed with 120.0 g of ethanol cosm. and 90.0 g of dem. water in a 2 l glass reactor. This initial charge was heated to 80° C. under a nitrogen-atmosphere. After the temperature had been reached, feeds 1 and 2 were started together. Feed 1 was metered in over the course of 3 hours and feed 2 was metered in over the course of 4 hours. The reaction mixture was further polymerized under reflux for 2 hours.

Feed 3 (0.90 g of sodium peroxodisulfate, 8.00 g of dem. water, 12.0 g of ethanol cosm.) was then metered in over the course of 30 min and, after-polymerized under reflux for 2 hours. Feed 4 (0.90 g of sodium peroxodisulfate, 8.00 g of dem. water, 12.0 g of ethanol cosm.) was then metered in over the course of 30 min and the mixture was after-polymerized under reflux again for 2 hours.

### Polymers L, M, N

Polymers L, M, N were synthesized analogously to polymer K.

The numbers which are given in the cells relating to the particular components are the percentages by weight of the particular component of the total amount of components i)+ii)+iii) used for the polymerization.

| | | Component ii) | | Comp. iii) | |
|---|---|---|---|---|---|
| Polymer | Comp. i) | MAA | AA | Monomer 2 | K value* |
| A | MMA | 10 | 10 | urethane acrylate (3) | 32.5 |
| | 78 | | | 2 | |
| B | MMA | 10 | 10 | Laromer ® UA 19T | 42.1 |
| | 78 | | | 2 | |
| C | MMA | 10 | 10 | — | — |
| | 80 | | | | |
| D | MMA | 10 | 10 | “urethane acrylate 3” | 32.1 |
| | 78 | | | 2 | |
| E | MMA | 10 | 10 | “urethane acrylate 1” | — |
| | 78 | | | 2 | |
| F | MMA | 10 | 10 | “urethane acrylate 1” | 33.7 |
| | 78 | | | 2 | |
| G | MMA | 10 | 10 | “urethane acrylate 1” | 33.5 |
| | 78 | | | 2 | |
| H | MMA | 12 | 12 | “acrylate 4”, | 35.5 |
| | 70 | | | 6 | |
| I | MMA | 10 | 10 | “urethane acrylate 1” | 36.6 |
| | 78 | | | 2 | |
| K | MMA | 10 | 10 | Ebecryl ® CL 1039, | 29.5 |
| | 76 | | | 4 | |
| L | MMA | 10 | 10 | “urethane acrylate 5” | 30.0 |
| | 76 | | | 4 | |
| M | MMA | 10 | 10 | “urethane acrylate 6” | 29.9 |
| | 76 | | | 4 | |
| N | MMA | 10 | 10 | “urethane acrylate 7” | 30.9 |
| | 76 | | | 4 | |

Residual monomer amount, determined after the polymerization and before steam distillation

### Determination of Residual Monomers

Monomers acrylic acid and methacrylic acid were determined by means of HPLC. For this, 0.2 to 1 g of the polymer solution were dissolved in 5 ml of methanol and precipitated out with 45 ml of 0.1% strength by weight H₃PO₄ by slow dropwise addition with stirring, then filtered through 0.2 μm Braunrand filters and injected into the HPLC apparatus.

Column: SymmetryShield® RP18 e from Waters
Eluent: water Milli-Q: H₃PO₄ conc.: methanol in the ratio 900 ml:1 ml:100 ml
UV detector: 205 nm

Monomeric MMA was determined by means of gas chromatography using an internal standard. For this, ca. 0.250 mg sample was treated with 2 ml of internal standard solution (1 mg of 1,4-dioxane in 2 ml of dimethylacetamide) and 1 ml of water. After dissolving the samples, analysis is made from the vapor space.

Separating column: DB 1 (100% dimethylpolysiloxane)

### Detector: FID

| | Unpolymerized | | |
|---|---|---|---|
| | component i) | Unpolymerized component ii) | |
| Polymer | [mg/kg]* | MAA [mg/kg]* | AA [mg/kg]* |
| A | 45 | 10 | 10 |
| B | 60 | 10 | 10 |
| C | 37 | <10 | <10 |
| D | 60 | 10 | 70 |
| E | 42 | 10 | 10 |
| F | 60 | 10 | 110 |
| G | | 10 | 110 |
| H | 35 | 10 | 160 |
| I | 6 | 10 | 60 |
| K | 60 | 10 | 40 |
| L | 70 | 10 | 40 |
| M | 55 | 10 | 30 |
| N | 90 | 10 | 20 |

*[mg/kg]: milligrams of unpolymerized component i) to ii) per kilogram of polymer

Application properties of the polymers produced according to the invention

| | | | Particle size | | | |
|---|---|---|---|---|---|---|
| | | | upon | | | |
| | | Setting^{b)}, | spraying a | | Ability to | |
| | | relative to | VOC 55 | Curl | be | |
| | Aerosol | Amphomer ® | aerosol | retention | washed | Stickiness |
| Polymer | appearance | LV 71 [%] | [mm]^{a)} | [%] | out | (Kempf) |
| D | clear | 100 | 33 | 84 | good | 0 |
| F | virtually | 101 | 36 | 90 | still good | 0 |
| | clear | | | | | |
| G | virtually | 92 | 36 | 83 | good | 0 |
| | clear | | | | | |
| H | slightly | 84 | 36 | 85 | good | 2 |
| | blue-tinged | | | | | |
| I | clear | 124 | 34 | 68 | still good | 0-1 |

^{a)}Particle size from VOC55 aerosol: 5% of the particular polymer already completely neutralized with AMP, 40% DME, 15% ethanol, 40% water; Spray device: spray head: Kosmos .020D vortex .018″ 21-6443-20, valve: DPV 33876 (Precision Valve)
^{b)}Setting: 3.0% strength by weight solution of the particular polymer already completely neutralized with AMP in ethanol/wasser (55:45 w/w)

The effects of different types of neutralizing agents and amounts of polymers were investigated below. Unless expressly mentioned otherwise, the following quantitative data are in % by weight.

| | | | Particle size | | | |
|---|---|---|---|---|---|---|
| | | | upon | | | |
| | | Setting^{d)}, | spraying a | | Ability to | |
| | | relative to | VOC 55 | Curl | be | |
| | Aerosol | Amphomer ® | aerosol | retention | washed | Stickiness |
| Polymer | appearance | LV 71 [%] | [mm]^{c)} | [%] | out | (Kempf) |
| I | clear | 108 | 40/41 | 68 | still good | 0 |

^{c)}Particle size from VOC55 aerosol: 5% of polymer I, 40% DME, 15% ethanol, 40% water, where, additionally, an amount of AMP required for complete neutralization of polymer I has been added Spray device: spray head: Kosmos .020D vortex .018″ 21-6443-20, valve: DPV 33876 (Precision Valve)
^{d)}Setting: 3.0% strength by weight solution of polymer I in ethanol/water (55:45 w/w), where, additionally, an amount of AMP required for complete neutralization of polymer I has been added.

| | | | Particle size | | | |
|---|---|---|---|---|---|---|
| | | | upon | | | |
| | | Setting, | spraying a | | Ability to | |
| | | relative to | VOC 55 | Curl | be | |
| | Aerosol | Amphomer ® | aerosol | retention | washed | Stickiness |
| Polymer | appearance | LV 71 [%]^{f)} | [mm]^{e)} | [%]^{e)} | out | (Kempf) |
| J | clear | 118 | 37 | 70 | good | 1 |

^{e)}Particle size from VOC55 aerosol: 5% of polymer J, 40% DME, 15% ethanol, 40% water, where, in addition, an amount of triethanolamine required for complete neutralization of polymer I has been added Spray device: spray head: Kosmos .020D vortex .018″ 21-6443-20, valve: DPV 33876 (Precision Valve)
^{f)}Setting: 3.0% strength by weight solution of polymer J in ethanol/water (55:45 w/w), where, additionally, an amount of triethanolamine required for complete neutralization of polymer J has been added.

| | | | Particle size | | | |
|---|---|---|---|---|---|---|
| | | | upon | | | |
| | | Setting, | spraying | | Ability to | |
| | | relative to | a VOC 55 | Curl | be | |
| | Aerosol | Amphomer ® | aerosol | retention | washed | Stickiness |
| Polymer | appearance | LV 71 [%]^{f)} | [mm]^{e)} | [%]^{e)} | out | (Kempf) |
| J | clear | 100 | 35 | 77 | good | 1 |

^{g)}Particle size from VOC55 aerosol: 5% of polymer J, 40% DME, 15% ethanol, 40% water, where, additionally, an amount of AMP required for complete neutralization of polymer J has been added Spray device: spray head: Kosmos .020D vortex .018″ 21-6443-20, valve: DPV 33876 (Precision Valve)
^{h)}Setting: 3.0% strength by weight solution of polymer J in ethanol/water (55:45 w/w), where additionally, an amount of AMP required for complete neutralization of polymer J has been added.

1. A method of producing polymers which compromise, in copolymerized
form,
i. 50-95% by weight of at least one ester of (meth)acrylic acid,
ii. 5-50% by weight of at least one olefinically unsaturated, free-radically polymerizable anionogenic or anionic compound and
iii. 0 to 30% by weight of at least one further free-radically polymerizable compound,
by free-radical polymerization in an alcohol-comprising solution, where the polymerization initiator used is at least one water-soluble initiator, wherein the polymerization solution comprises water in the range from more than 25 to 50% by weight, wherein component i) is chosen from the group consisting of methyl (meth)acrylate, ethyl (meth)acrylate, n-propyl (meth)acrylate, isopropyl (meth)acrylate, methyl ethacrylate, ethyl ethacrylate, n-propyl ethacrylate, isopropyl ethacrylate, n-butyl ethacrylate, tert-butyl ethacrylate, isobutyl ethacrylate, n-butyl (meth)acrylate, tert-butyl (meth)acrylate, isobutyl (meth)acrylate, sec-butyl (meth)acrylate, 2-pentyl (meth)acrylate, 3-pentyl (meth)acrylate, isopentyl acrylate, neopentyl acrylate, n-octyl (meth)acrylate, 1,1,3,3-tetramethylbutyl (meth)acrylate, ethylhexyl (meth)acrylate, n-nonyl (meth)acrylate, n-decyl (meth)acrylate, n-undecyl (meth)acrylate, tridecyl (meth)acrylate, myristyl (meth)acrylate, pentadecyl (meth)acrylate, palmityl (meth)acrylate, heptadecyl (meth)acrylate, nonadecyl (meth)acrylate, arachinyl (meth)acrylate, behenyl (meth)acrylate, lignocerenyl (meth)acrylate, cerotinyl (meth)acrylate, melissinyl (meth)acrylate, palmitoleinyl (meth)acrylate, oleyl (meth)acrylate, linoleyl (meth)acrylate, linolenyl (meth)acrylate, stearyl (meth)acrylate, lauryl (meth)acrylate, phenoxyethyl acrylate, t-butylcyclohexyl acrylate, cyclohexyl (meth)acrylate, ureido (meth)acrylate, tetrahydrofurfuryl (meth)acrylate and mixtures thereof.

2. The method according to claim 1, wherein the polymerization solution comprises at most 50% by weight of at least one alcohol, and the total amount of water and alcohol is at most 95% by weight of the polymerization solution.

3. The method according to claim 1, wherein the polymerization solution comprises water in the range from 28 to 40% by weight and alcohol in the range of 30 to 40% by weight.

4. The method according to claim 2, wherein the at least one alcohol is or comprises ethanol and/or isopropanol.

5. The method according to claim 1, wherein the water-soluble polymerization initiator is chosen from the group consisting of peroxides, hydroperoxides, peroxodisulfates, percarbonates, peroxide esters, azo compounds and mixtures thereof.

6. The method according to claim 1, wherein the water-soluble polymerization initiator is chosen from the group consisting of hydrogen peroxide, lithium peroxodisulfate, sodium peroxodisulfate, potassium peroxodisulfate, ammonium peroxodisulfate and mixtures thereof.

7. The method according to claim 1, wherein the polymerization initiator is chosen from the group consisting of
2,2′-azobis[2-(5-methyl-2-imidazolin-2-yl)propane]dihydrochloride,
2,2′-azobis[2-(2-imidazolin-2-yl)propane]dihydrochloride,
2,2′-azobis[2-(2-imidazolin-2-yl)propane]disulfate dihydrate,
2,2′-azobis(2-methylpropionamide) dihydrochloride,
2,2′-azobis[N-(2-carboxyethyl)-2-methylpropionamidine]tetrahydrate,
2,2′-azobis[2-(3,4,5,6-tetrahydropyrimidin-2-yl)propane]dihydrochloride,
2,2′-azobis{2-[1-(2-hydroxyethyl)-2-imidazolin-2-yl]propane}dihydrochloride,
2,2′-azobis[2-(2-imidazolin-2-yl)propane],
2,2′-azobis{2-methyl-N-[1,1-bis(hydroxymethyl)-2-hydroxyethyl]propionamide,
2,2′-azobis {2-methyl-N-[2-(1-hydroxybutyl)]propionamide},
2,2′-azobis[2-methyl-N-(2-hydroxyethyl)propionamide] and their mixtures thereof.

8. The method according to claim 1, wherein the temperature at which the polymerization is carried out is in the range of 30 to 120° C.

9. The method according to claim 1, wherein the polymerization is carried out under a pressure in the range from 2 to 10 bar.

10. The method according to claim 1, wherein component i) is chosen from the group consisting of methyl methacrylate, ethyl methacrylate, n-propyl methacrylate, isopropyl methacrylate, n-butyl methacrylate, tert-butyl methacrylate, isobutyl methacrylate, sec-butyl methacrylate and mixtures thereof.

11. The method according to claim 1, wherein component ii) is chosen from the group consisting of acrylic acid, methacrylic acid, ethacrylic acid, alpha-chloroacrylic acid, crotonic acid, maleic acid, maleic anhydride, itaconic acid, citraconic acid, mesaconic acid, glutaconic acid, aconitic acid, fumaric acid, half-esters of monoethylenically unsaturated dicarboxylic acids having 4 to 10 carbon atoms.

12. The method according to claim 1, wherein component iii) is chosen from urethane-group-containing esters of (meth)acrylic acid.

13. A polymer obtainable by a method according to claim 1.

14. The method of producing cosmetic preparations comprising preparing a polymer according to claim 13.

15. A cosmetic preparation comprising at least one polymer according to claim 13.

16. The method according to claim 11, wherein the half-esters of monoethylenically unsaturated dicarboxylic acids have 4 to 6 carbon atoms.

17. The method according to claim 2, wherein the polymerization solution comprises water in the range from 28 to 40% by weight and alcohol in the range of 30 to 40% by weight.

18. The method according to claim 3, wherein the at least one alcohol is or comprises ethanol and/or isopropanol.

19. The method according to claim 2, wherein the water-soluble polymerization initiator is chosen from the group consisting of peroxides, hydroperoxides, peroxodisulfates, percarbonates, peroxide esters, azo compounds and mixtures thereof.

20. The method according to claim 3, wherein the water-soluble polymerization initiator is chosen from the group consisting of peroxides, hydroperoxides, peroxodisulfates, percarbonates, peroxide esters, azo compounds and mixtures thereof.
